SEQUENCE LISTING (2 sequences)

SEQ ID NO: 1 - protein, 130 aa, Homo sapiens
KVFERCELARTLKRLGMDGYRGISLANWMCLAKWESGYNTRATNYNAGDRSTDYGIFQINSRYWCNDGKTPGAVNACHLSCSALLQDNIADAVACAKRVVRDPQGIRAWVAWRNRCQNRDVRQYVQGCGV

SEQ ID NO: 2 - protein, 5 aa, Homo sapiens
  UNSURE (1)..(1): Xaa represents unknown amino acid
XVFER

(19) United States
(12) Patent Application Publication
(10) Pub. No.: US 2012/0028881 A1
(43) Pub. Date: Feb. 2, 2012

(54) METHODS AND REAGENTS FOR DIAGNOSING ATHEROMA

(75) Inventors: Martin R. Wilkins, London (GB); Robert John Edwards, London (GB)

(73) Assignee: IMPERIAL INNOVATIONS LIMITED, London (GB)

(21) Appl. No.: 13/132,828

(22) PCT Filed: Dec. 3, 2009
(86) PCT No.: PCT/GB2009/002821

§ 371 Date: Oct. 12, 2011

(30) Foreign Application Priority Data
Dec. 4, 2008 (GB) 0822140.0

(51) Int. Cl. A61K 38/28; A61P 9/10; A61P 7/02; C40B 30/04; A61P 3/06; G01N 33/92; G01N 33/88; G01N 33/573; A61P 3/10

(52) U.S. Cl. 514/1.9; 435/7.4; 506/9

(57) ABSTRACT

The invention provides a method of diagnosing atheroma in an individual, the method comprising providing a sample from the individual, determining the level of lysozyme in the sample and assessing whether the level is indicative of atheroma in the individual. The invention also provides the use of a reagent which selectively identifies lysozyme in the assessment of atheroma in an individual.

The present invention relates to methods and reagents for use in relation to the diagnosis and prognosis of atheroma in a patient, preferably a human patient.

The listing or discussion of a prior-published document in this specification should not necessarily be taken as an acknowledgement that the document is part of the state of the art or is common general knowledge.

Atheroma refers to a mass of plaque of degenerated, thickened arterial intima occurring in atherosclerosis. Atheroma develops preferentially in subjects presenting biochemical risk factors including smoking, hypertension, diabetes mellitus, hypercholesterolemia, elevated plasma low-density lipoprotein (LDL) and triglycerides, hyperfibrinogenemia, raised lipoprotein A, and raised homocysteine, among others. It decreases blood flow and might cause ischemia and tissue destruction in organs supplied by the affected vessel. Atheromatous plaques develop over a number of decades in humans, leading to complications such as coronary and cerebral ischemic and thromboembolic diseases, myocardial and cerebral infarction, and peripheral vascular disease.

Atheroma develops between the endothelial lining and the smooth muscle wall region (media) of the arterial tube. In the early stages of development, plaques are composed largely of white blood cells, particularly macrophages that have taken up oxidised LDL. Later, these macrophages mature into foam cells. When foam cells die, their contents are released attracting more macrophages and creating an extracellular lipid core near the centre to inner surface of the atheroma. Conversely, the outer older portions of the plaque become more calcific, less metabolically active and more physically stiff over time.

Atheroma is the root cause of various cardiovascular diseases such as angina, heart attack, stroke and peripheral vascular disease. It is one of the most important causes of death in the developed world and its incidence is increasing in the developing world. While it is possible to assign risk to any given individual based on population risk factors such as blood pressure and cholesterol, the diagnosis of established disease generally requires an invasive test. There is no blood test for atheroma at the present time.

A prevalent form of atheroma, in the coronary artery, leads to coronary artery disease (CAD).

CAD is the most common type of heart disease and is the leading cause of death in the developed and developing world (1). Present prevalence trends suggest that remaining lifetime risk for healthy 40-year-old men or women in the United States is 50% and 33%, respectively (2). Individuals affected with CAD have limited exercise capacity, physical debility and chronic stress that diminish the quality of life. Early detection and accurate disease stratification of CAD is therefore highly desirable.

Diagnosis of CAD is difficult in patients presenting with chest pain alone as they indicate a plethora of cardiac conditions. While it is possible to assign risk to any given individual, based on population risk factors such as blood pressure, cholesterol levels and smoking habits, the diagnosis of established disease requires an invasive coronary angiography (CAG) test (3). CAG allows direct visualization of coronary anatomy and assessment of the extent and prognostic implications of CAD. However it is invasive with a small risk of serious complications and requires a dedicated infrastructure and qualified staff which makes it unsuitable as a screening test. Initial risk assessment of patients with suspected CAD recommend clinical evaluation and exercise stress testing (ETT) as the first step in diagnosis and risk stratification (4, 5). However, meta-analyses have shown that ETT has a sensitivity of only 65-70% and a specificity of only 70-75% (6, 7). It is not surprising, therefore, that 15-20% of patients referred for coronary angiography following initial evaluation and ETT have no sign of atheromatous coronary disease (8).

Other non-invasive methods of diagnosis such as myocardial perfusion scanning and stress echocardiography are also available (sensitivity 75-85%, specificity 85-90%) (9). CT coronary angiography using multi-detector row CT (MDCT) appears to have the best diagnostic accuracy (sensitivity 85-90%, specificity 85-95%) (10, 11), although the accuracy varies depending on patient and lesion characteristics (10, 12).

The availability of tests to diagnose atheroma is limited by requirements for special training and expensive equipment. Since there is no available blood test to detect the presence of atheroma, development of a simple test that would improve the accuracy of non invasive screening modalities is needed as a reliable indicator which may help to prognosticate the extent and severity of atheroma as well as indicate the likelihood of future events. Classification of the risk in this way should lead to better management of patients and will be cost effective.

Understanding the pathogenesis of atheroma as well as advances in molecular analytical technology, has led to research into ‘biomarkers’ which may help in early risk stratification and prognostication. Studies have included genotype analysis (13, 14), single nucleotide polymorphisms (15, 16) and post translational modifications (17-19) in patients with CAD, as well as circulating biomarkers in unstable CAD (20), markers of disease progression in stable angina (21, 22) and in asymptomatic CAD (23). However, none of these studies have identified an accurate marker for atheroma that can be used as the basis for a diagnostic blood test. The inventors have now surprisingly and unexpectedly found a strong correlation between lysozyme levels in plasma and the presence of atheroma, such that it may be used as a biomarker of atheroma.

Accordingly, a first aspect of the invention provides a method of diagnosing atheroma in an individual, the method comprising providing a sample from the individual, determining the level of lysozyme in the sample and assessing whether the level is indicative of atheroma in the individual.

It will be appreciated that the invention includes a method of assessing whether an individual is suffering from atheroma, the method comprising providing a sample from the individual, determining the level of lysozyme in the sample and assessing whether the level is indicative of atheroma in the individual. It will be appreciated that this assessment may aid diagnosis, and may be used in association with other tests, or observations by the physician, in reaching a diagnosis.

Typically, the method is used to determine whether the presenting symptoms of an individual (such as chest pain, shortness of breath, claudication and weakness) are due to widespread atheroma within the circulation of that individual. Further, the method may be used to screen individuals at risk of atheroma and may aid in the prioritisation of invasive investigations and/or interventions such as angiography and angioplasty. For example the individual may have one or more risk factors of atheroma, including, for example, obesity, smoking, hypertension, diabetes mellitus, hypercholesterolemia, elevated plasma LDL and triglycerides, hyperfibrinogenemia and hyperglycemia. Thus it will be appreciated that the method may be used to single out individuals for more invasive tests with greater efficiency. Further, the method will allow identification of individuals at greater risk of future events such as myocardial infarction and stroke. The development of atheroma is also associated with age and so, typically, the individual is over 40, 45, 50, 55, 60, 65, 70, 75, 80, 85 or 90 years of age.

The individual may be a human or mammalian individual, such as a horse, dog, pig, cow, sheep, rat, mouse, guinea pig or primate. Preferably, the individual is a human individual.

Atheroma may be in any one or more of the aorta, a coronary artery, a renal artery, a neck artery, a cerebral artery, a limb artery (eg leg, arm, shoulder or toe) or visceral artery. Preferably, atheroma is in a coronary artery.

By ‘lysozyme’ we include human lysozyme, the amino sequence of which is provided in FIG. 3 (SEQ ID No: 1). However, it is well known that certain polypeptides are polymorphic, and it is appreciated that some natural variation of this sequence may occur. Thus, in an embodiment, the invention is not limited to determining the level of human lysozyme having the sequence listed in FIG. 3 (SEQ ID No: 1), but includes naturally occurring variants thereof in which one or more of the amino acid residues have been replaced with another amino acid. The invention also includes determining the level of lysozyme in other species which have an orthologous sequence to that in FIG. 3 (SEQ ID No: 1), for example lysozyme from horse, dog, pig, cow, sheep, rat, mouse, guinea pig or a primate. It will be appreciated, that when the sample is taken from a particular individual, the lysozyme whose level is determined is preferably that of the same species as the individual. Thus, when the individual is human, the level of human lysozyme is measured, and so on.

The level of lysozyme which is indicative of atheroma in the individual may vary depending on the type of individual (eg human, horse, dog and so on). The level may be determined by comparing the levels in known atheroma individuals with those in normal individuals (ie those with no sign or symptoms of atheroma). Typically, the level which is indicative of atheroma is a level which is greater than 1 standard deviation above the mean level of lysozyme in a healthy population, for example greater than 1.5, 2, 2.5, 3, 3.5, 4, 4.5, 5, 5.5, 6, 6.5, 7, 7.5 or 8 standard deviations above the mean level of lysozyme in a healthy population, that is a population of normal individuals (ie in individuals with no signs or symptoms of atheroma). Preferably, the population comprises at least 5, 10, 50, 100, 200, 300, 400 or 500 individuals and more preferably at least 1000 individuals. It is preferred if the normal individuals are assessed using the most sensitive and specific detection techniques for atheroma available (eg angiography, CT coronary angiography using multidetector row CT (MDCT), transesophageal echocardiography, magnetic resonance imaging, high resolution computerised tomography scanning and/or Doppler ultrasound of arterieis) other than the methods of the present invention and confirmed not to have atheroma (eg no evidence of atheroma is detected at any position in the coronary artery). Preferably, the healthy population is a population of individuals that have been shown not to have atheroma, and thereafter have been shown not to develop atheroma using the same techniques, for example, for at least 6 months, or 1, 2, 3, 4 or 5 years, or more. In addition, it is preferred if the ‘normal individuals’ have no or few risk factors for atheroma including, for example, obesity, smoking, hypertension, diabetes mellitus, hypercholesterolemia, elevated plasma LDL and triglycerides, hyperfibrinogenemis and hyperglycamia.

The sample from the individual may be any suitable sample. In particular embodiments of the invention, a suitable sample is obtained from the individual who is to be assessed (eg diagnosed or prognosed), and this sample is provided for analysis of the level of lysozyme. Conveniently, the sample is a fluid sample and it may be blood, serum, plasma, urine or saliva. The sample may comprise white blood cells and the lysozyme content in white blood cells measured. It is particularly convenient if the sample is a plasma sample which may be prepared from a blood sample in a standard way (for example by collection in citrate tubes followed by centrifugation). It will be appreciated that the blood may be arterial, venous or capillary blood. Thus the sample may be arterial, venous or capillary blood, serum or plasma. Arterial blood may be obtained, for example, from a patient undergoing angiography from a catheter inserted into an artery. Venous blood is routinely collected using a hypodermic needle whereas capillary blood is routinely sampled by finger pricking.

As described in Example 1, the inventors have correlated arterial plasma lysozyme levels with atheroma in patients whose coronary arteries were examined and where it was found that one (1VD), two (2VD) or three (3VD) vessels were affected. By “affected” we mean occluded by more than two-thirds of the vessel cross-section at the site of maximum occlusion, when assessed by angiography. Thus, in a particularly preferred embodiment, the method is used to diagnose atheroma in a coronary artery. Typically, the level of lysozyme in arterial blood, serum or plasma which is indicative of atheroma wherein at least one vessel is affected is a level which is greater than 1 standard deviation above the mean level in a healthy population; the level of lysozyme in arterial blood, serum or plasma which is indicative of atheroma wherein at least two vessels are affected is a level which is greater than 1 standard deviation above the mean level in a healthy population; and the level of lysozyme in arterial blood, serum or plasma which is indicative of atheroma wherein at least three vessels are affected is a level which is greater than 7.5 standard deviations above the mean level in a healthy population. These cut-off values were determined from receiver-operator characteristic curve analyses of these data and were values that gave optimum separations between the respective groups of patients.

Using these cut-off values, when comparing normal individuals with 1VD patients the false positive rate is 20% and the false negative rate is 9%, when comparing normal individuals with 2VD patients, the false positive rate is 0% and the false negative rate is 9%, and when comparing normal individuals with 3VD patients, both the false positive and false negative rates are 0%. The rate of false positives and negatives give an indication of sensitivity (% of patients diagnosed correctly as positive) and specificity (% normal patients diagnosed correctly as negative) and can be derived from receiver-operator characteristic curves as shown in Table 2. It will be appreciated that different cut-off values lead to different false positive and false negative rates.

As can be seen from Example 1, there is a degree of overlap in the lysozyme levels in arterial plasma in patients with 1VD and those in patients with 2VD, such that the level of lysozyme which is indicative of atheroma in a patient with 1VD is the same as that which is indicative of atheroma in a patient with 2VD. Nevertheless, lysozyme levels in arterial plasma are higher in patients with 2VD than those in patients with 1VD, as reflected in the improved ability of the test for 2VD compared to 1VD, the test for 2VD having a 0% rate of false positives whereas the test for 1VD has a rate of 20%.

The study in Example 1 also correlated venous plasma lysozyme levels with atheroma in coronary arteries wherein at least three (3VD) vessels are affected. The level of lysozyme in venous plasma in patients with 3VD (mean 2.15 μg/mL and SD 1.64 μg/mL) was not as high as that in arterial plasma. Using a cut-off of 1 standard deviation above the mean level of lysozyme in venous plasma in a healthy population, when comparing normal individuals with 3VD patients, the false positive rate is 53% and the false negative rate is 19%. Thus in an embodiment, the sample may be venous blood, serum or plasma and the level of lysozyme which is indicative of atheroma in a coronary artery wherein at least three vessels are affected is a level which is greater than 1 standard deviation above the mean level in a healthy population.

As described in Example 1, the inventors measured the level of lysozyme in human plasma by an enzyme immunoassay (EIA) using the Biomedical Technologies Lysozyme Assay kit (details provided in Appendix 1), wherein the sample was diluted in phosphate-buffered saline by a factor of 1:1000 prior to measurement. Arterial plasma levels of lysozyme greater than 1.5 μg/mL were typically indicative of atheroma in a coronary artery wherein at least one vessel was affected (ie occluded by more than two-thirds when assessed by angiography). Using the same assay, arterial plasma levels of lysozyme greater than 1.5 μg/mL were typically indicative of atheroma in a coronary artery wherein at least two vessels were affected, and arterial plasma levels of lysozyme greater than 5.0 μg/mL were typically indicative of atheroma in a coronary artery wherein three vessels were affected. Further, using the same assay, venous plasma levels of lysozyme greater than 1.5 μg/mL were typically indicative of atheroma in a coronary artery wherein three vessels were affected.

Accordingly, the invention includes a method of diagnosing atheroma in an individual, the method comprising providing a sample from the individual and determining the level of lysozyme in the sample, wherein a level of lysozyme in the sample greater than 1.5 μg/mL is indicative of atheroma. Conveniently, the value of 1.5 μg/mL corresponds to the amount of lysozyme in the sample that gives rise to a measurement of 1.5 μg/mL lysozyme if measured by an enzyme immunoassay (EIA) using the Biomedical Technologies Lysozyme Assay kit wherein the sample is diluted in phosphate-buffered saline by a factor of 1:1000 prior to measurement.

Typically, when the sample is arterial blood, serum or plasma a lysozyme level greater than 1.5 μg/mL, if measured as above, is indicative of atheroma in a coronary artery wherein at least one vessel is affected (ie occluded by more than two-thirds when assessed by angiography).

The invention also includes a method of diagnosing a coronary atheroma in an individual, the method comprising providing a sample of arterial blood, serum or plasma from the individual and determining the level of lysozyme in the sample, wherein a level of lysozyme in the sample greater than 1.5 μg/mL is indicative of atheroma in a coronary artery wherein at least two vessels are occluded by more than two-thirds when assessed by angiography. Conveniently, the value of 1.5 μg/mL corresponds to the amount of lysozyme in the sample that gives rise to a measurement of 1.5 μg/mL lysozyme if measured by an enzyme immunoassay (EIA) using the Biomedical Technologies Lysozyme Assay kit wherein the sample is diluted in phosphate-buffered saline by a factor of 1:1000 prior to measurement.

The invention also includes a method of diagnosing a coronary atheroma in an individual, the method comprising providing a sample of arterial blood, serum or plasma from the individual and determining the level of lysozyme in the sample, wherein a level of lysozyme in the sample greater than 5.0 μg/mL is indicative of atheroma in a coronary artery wherein three vessels are occluded by more than two-thirds when assessed by angiography. Conveniently, the value of 5.0 μg/mL corresponds to the amount of lysozyme in the sample that gives rise to a measurement of 5.0 μg/mL lysozyme if measured by an enzyme immunoassay (EIA) using the Biomedical Technologies Lysozyme Assay kit wherein the sample is diluted in phosphate-buffered saline by a factor of 1:1000 prior to measurement.

The invention also includes a method of diagnosing a coronary atheroma in an individual, the method comprising providing a sample of venous blood, serum or plasma from the individual and determining the level of lysozyme in the sample, wherein a level of lysozyme in the sample greater than 1.5 μg/mL is indicative of atheroma in a coronary artery wherein three vessels are occluded by more than two-thirds when assessed by angiography. Conveniently, the value of 1.5 μg/mL corresponds to the amount of lysozyme in the sample that gives rise to a measurement of 1.5 μg/mL lysozyme if measured by an enzyme immunoassay (EIA) using the Biomedical Technologies Lysozyme Assay kit wherein the sample is diluted in phosphate-buffered saline by a factor of 1:1000 prior to measurement.

By ‘if measured’ we mean that had the lysozyme level been determined by an enzyme immunoassay (EIA) using the Biomedical Technologies Inc Lysozyme Assay kit wherein the sample is diluted in phosphate-buffered saline by a factor of 1:1000 prior to measurement, it would give a particular value. However, the skilled person will appreciate that many other methods may be used to determine the level of lysozyme in a sample. The particular value determined by an enzyme immunoassay (EIA) using the Biomedical Technologies Inc Lysozyme Assay kit wherein the sample is diluted in phosphate-buffered saline by a factor of 1:1000 prior to measurement, may correspond to a different value had the lysozyme level been determined according to another method. Thus, the invention includes methods of diagnosing a coronary atheroma in an individual, the methods comprising providing a sample from the individual and determining the level of lysozyme in the sample according to any method. The level of lysozyme that is indicative of atheroma in a coronary artery as defined above is one that corresponds to a level greater than any of 1.5 μg/mL or 5.0 μg/mL if the level is determined by an enzyme immunoassay (EIA) using the Biomedical Technologies Inc Lysozyme Assay kit wherein the sample is diluted in phosphate-buffered saline by a factor of 1:1000 prior to measurement.

The above lysozyme concentrations of 1.5 μg/mL and 5.0 μg/mL correspond to the concentration of lysozyme in plasma. It will be appreciated that when the sample analysed is blood, the values may need to be revised to reflect any volume differences between plasma and blood resulting from the presence of cells.

It will be appreciated that diagnosing atheroma in a patient may be predictive of the individual developing a disease or a disorder occurring as a direct result of atheroma. Depending on where the atheroma is situated, the person may go on to develop any of, for example, coronary artery disease, stroke, walking difficulty, gangrene infection or aneurysm formation. For example, identifying an individual with a slightly raised level of lysozyme relative to normal individuals (indicating a small amount of atheroma), may be used to predict the likelihood of that individual developing coronary artery disease. Accordingly, by ‘method of diagnosing atheroma in an individual’ we also include a method a predicting a disease or a disorder occurring as a direct result of the atheroma.

A second aspect of the invention provides a method of prognosing atheroma in an individual who has atheroma, the method comprising providing a sample from the individual, determining the level of lysozyme in the sample and assessing whether the level is indicative of a particular outcome for the individual.

The individual may be a human or mammalian individual such as a horse, dog, pig, cow or sheep. Preferably, the individual is a human individual.

The atheroma may be in any one or more of the aorta, a coronary artery, a renal artery, a neck artery, a cerebral artery, a limb artery (eg leg, arm, shoulder or toe) or a viscera artery. Preferably, the atheroma is in a coronary artery.

Preferences for the sample are defined above with respect to the first aspect of the invention.

It will be appreciated that the invention includes a method of assessing the likely outcome of an individual who has atheroma, the method comprising providing a sample from the individual, determining the level of lysozyme in the sample and assessing whether the level is indicative of a particular outcome for the individual. For example, when the individual has 1VD coronary artery disease, the method may be used to assess whether that individual will go on to develop 3VD coronary artery disease. It will be appreciated that this assessment may aid prognosis, for example in follow up clinics, and may be used in association with other tests, or observations by the physician, in reaching a prognosis. Further, the method may be used as an indication for the need for more aggressive therapy.

Atheroma may have already been diagnosed in the individual. For example, atheroma in a coronary artery may have been detected by CT coronary angiography using multidetector row CT (MDCT). Advanced atheroma of the aorta may have been identified by transesophageal echocardiography. Additionally or alternatively, atheroma may have been diagnosed using magnetic resonance imaging, high resolution computerised tomography scanning and Doppler ultrasound of arteries. Further, the individual may have been diagnosed with a disease or a disorder occurring as a direct result of the atheroma. In the case of a coronary atheroma, the individual may have been diagnosed with CAD using, for example, coronary angiography or exercise stress testing. In the case of aortic, neck or cerebral atheroma, the individual may have suffered a stroke. Atheroma in the leg may be manifested as walking difficulty while atheroma in distal limb arteries may cause gangrene infection of the extremities. It will be appreciated that atheroma in any major artery may also lead to aneurysm formation. Thus, the individual may have been diagnosed with atheroma and/or present with diseases or disorders associated with atheroma. Typically, the individual is over 40, 45, 50, 55, 60, 65, 70, 75, 80, 85 or 90 years of age.

The level of lysozyme which is indicative of a poor prognosis may vary depending on the type of individual. The level may be determined by comparing levels in atheroma patients who fare well and those that have a poor outcome. For example, by following the progression of atheroma in individuals it will be possible to relate lysozyme levels to clinical outcomes. Typically, a lysozyme level which is greater than 2 standard deviations (SD) above the mean level of lysozyme in a population of individuals with atheroma who fare well is indicative of a poor outcome, for example a level which is greater than 2.5, 3, 3.5, 4, 4.5, 5, 5.5, 6, 6.5, 7, 7.5 or 8 standard deviations above the mean. Preferably, the population of individuals comprises at least 5, 10, 50, 100, 200, 300, 400 or 500 individuals, and more preferably at least 1000 individuals.

By a population of individuals with atheroma who fare well we mean individuals who have been diagnosed with atheroma using one the techniques described above other than the method of the invention, and who have survived for a period of at least 6 months and more preferably for at least 1, 2, 3, 4 or 5 years.

As discussed above, the inventors have correlated an arterial plasma lysozyme level above 5.0 μg/mL and a venous plasma lysozyme level above 1.5 μg/mL with atheroma in a coronary artery wherein three vessels are affected, ie an example of a poor prognosis which if untreated would put the individual at high risk of death. Accordingly, the invention includes a method of prognosing atheroma in an individual who has atheroma, the method comprising providing a sample from the individual and assessing whether the level is indicative of a particular outcome for the individual, wherein a level of lysozyme in the sample greater than 1.5 μg/mL is indicative of a poor outcome. Typically, when the sample is arterial blood, serum or plasma, a level of lysozyme in the sample greater than 5.0 μg/mL is indicative of a poor outcome and when the sample is venous blood, serum or plasma, a level of lysozyme in the sample greater than 1.5 μg/mL is indicative of a poor outcome. Conveniently, the value of 1.5 μg/mL or 5.0 μg/mL corresponds to the amount of lysozyme in the sample that gives rise to a measurement of 1.5 μg/mL or 5.0 μg/mL lysozyme respectively, if measured by an enzyme immunoassay (EIA) using the Biomedical Technologies Lysozyme Assay kit wherein the sample is diluted in phosphate-buffered saline by a factor of 1:1000 prior to measurement.

It will be appreciated that if the prognosis is of a poor outcome, the physician will be able to tailor treatment of the individual accordingly. Such treatments may include balloon angioplasty and/or insertion of a stent, bypass surgery using nativel conduits (arteries or veins) or artificial conduits and/or endoterectomy to remove the atheroma from the lining of the artery. Similarly, if the prognosis is of a good outcome, the physician will be able to tailor treatment of the individual accordingly.

Preferably, the ‘level of lysozyme’ measured is the level of total lysozyme in the sample regardless of activity. For example, the level of lysozyme may include both active and inactive forms of the enzyme.

The plasma lysozyme activity measurements shown in Example 1 indicate that the majority of the lysozyme detected in the plasma of patients with 3VD was partially or completely inactive. Thus, it is appreciated that the level of only the inactive form of the enzyme may be determined. It is believed that the lysozyme derived from plaques is largely inactive but will still contain a proportion of active lysozyme. Thus, it is appreciated that the level of only the active form of the enzyme may be determined. Preferably, when the sample taken from the individual is from an artery or capillary, the level of total lysozyme regardless of activity is determined or the level of inactive lysozyme is determined. Since the active lysozyme may persist longer in blood, it is likely that active lysozyme is the predominant form detected in venous samples. Thus, when the sample taken from the individual is from a vein, the level of active lysozyme is preferably determined.

The level of lysozyme may be measured in the sample by any suitable means. One convenient way of measuring the level of lysozyme in the sample is to make use of a reagent which can identify lysozyme. Conveniently, the reagent is one which binds to lysozyme, but it may be any other type of suitable reagent. It is appreciated that since active and/or inactive forms of lysozyme may be detected, the reagent that is used may bind to active and/or inactive forms of lysozyme depending on the forms of lysozyme being detected. For example, if only inactive lysozyme is being detected and inactivation is by myeloperoxidase, the reagent may be one that binds to the chemically modified adducted form of lysozyme.

HPLC, gel electrophoresis and capillary electrophoresis, followed by UV or fluorescent detection, may be used to detect and quantify lysozyme. Such techniques may be used to separate and quantify both enzymatically active and inactive forms of lysozyme.

Reagents which bind to lysozyme include antibodies and peptides, for example those selected from a combinatorial or phage display library. By the term “antibodies” we include whole antibodies which bind to lysozyme but also fragments of antibodies which bind lysozyme such as Fv, Fab and F(ab)₂ fragments as well synthetic antibodies or antibody fragments such as single chain Fv (scFv) molecules and domain antibodies (dAbs). The antibody fragments and synthetic antibodies retain antigen binding activity (and usually contain some or all of the complementarity determining regions (CDRs) of a parent antibody molecule). Antibodies that bind lysozyme selectively are known in the art and are commercially available, for example Biomedical Technologies Inc (Stoughton, USA) supplies an enzyme-linked immunosorbent assay (ELISA) kit which contains antibodies to human lysozyme. It will be appreciated that the antibody is typically one which has been raised to or selected using human lysozyme. In any event, antibodies for lysozyme may be made using well known technology such as the hybridoma method for making monoclonal antibodies, and phage display techniques for making synthetic antibody fragments. Suitable methods for the production and use of antibodies are described and referred to in “Using antibodies: A laboratory manual”, Ed Harlow and David Lane, Cold Spring Harbor Press, Cold Spring Harbor, N.Y., 1999.

Preferably, antibodies which bind preferentially to lysozyme compared to other molecules in the sample from the individual (eg plasma) are used. Preferably, the antibody has at least a 10 fold-higher affinity for lysozyme than for any other component in the sample and more preferably at least a 50 fold, 100 fold, 500 fold, 1000 fold or 10000 fold-higher affinity.

Conveniently, the level of lysozyme in the sample is measured using an immunoassay. The antibody selective for lysozyme may itself be labelled, for example with a radioactive label or a fluorescence label or with an enzyme. Alternatively, and preferably, it is detected with a secondary antibody, which binds the antibody selective for lysozyme which is labelled. It is particularly convenient if the immunoassay is an ELISA. Immunoassays are well known in the art (see, for example, Immunoassays: A practical approach. James P. Gosling (ed), Oxford University Press, 2000, ISB4 0-19-963710-5), and, as described above, an ELISA for human lysozyme is commercially available. In particular, a suitable assay for lysozyme is sold by Biomedical Technologies Inc, Stoughton, USA. It is particularly preferred if the lysozyme in the sample is measured by an enzyme immunoassay (EIA) using the Biomedical Technologies Lysozyme Assay kit wherein the sample is diluted in phosphate-buffered saline by a factor of 1:1000 prior to measurement.

Other methods of measuring lysozyme in a sample are available. For example, as discussed in more detail in the Examples, mass spectrometry may be used since human lysozyme has a characteristic molecular ion signature in plasma samples analysed by SELDI-TOF mass spectrometry corresponding to an m/z of 14735 (a value has an accuracy of approximately 0.2% when determined by SELDI-TOF MS). There is also a minor peak at m/z 7375. It will be understood that lysozymes from other species will vary in mass and therefore will have a different characteristic m/z. Methods of measuring the levels of compounds in a sample by mass spectrometry are well known in the art and any suitable form of mass spectrometry may be used (see, for example, Mass Spectrometry Principles and Applications, E. De Hoffmann, J. Charette, V. Stroobant, Wiley & Sons, New York, N.Y., 1996).

Another means of determining the level of lysozyme in a sample is to measure the activity of lysozyme in the sample. As discussed in Example 1, the majority of lysozyme detected in the plasma of patients with 3VD was partially or completely inactive. In terms of specific activity (ie ratio of activity to quantity), lysozyme from patients with 3VD had significantly less activity than that from normal patients. Accordingly, the level of lysozyme in a sample may be determined by measuring the specific activity of lysozyme. For example, samples taken from individuals with atheroma will have higher levels of lysozyme which correspond to a lower specific activity of lysozyme, relative to samples taken from normal individuals. Typically, the specific activity which is indicative of atheroma is an activity which is less than 1 standard deviation below the mean specific activity of lysozyme in a healthy population, for example less than 1.5, 2, 2.5, 3, 3.5, 4, 4.5, 5, 5.5, 6, 6.5, 7, 7.5 or 8 standard deviations below the mean. Similarly, samples taken from individuals with atheroma who have a poor prognosis have higher levels of lysozyme which correspond to a lower specific activity of lysozyme, relative to samples taken from individuals with atheroma who fare well. Typically, the specific activity which is indicative of a poor outcome, is an activity which is less than 1 standard deviation below the mean specific activity of lysozyme in a population of individuals with atheroma who fare well, for example less than 1.5, 2, 2.5, 3, 3.5, 4, 4.5, 5, 5.5, 6, 6.5, 7, 7.5 or 8 standard deviations below the mean. The activity of lysozyme may be assayed using any suitable enzyme assay known in the art including, for example, that used in Example 1. It will be appreciated that when determining the specific activity of lysozyme both the total amount of lysozyme regardless of activity is to be measured (eg by EIA) and the activity of lysozyme is to be measured (eg by an enzymatic-based measurement).

In a further embodiment of the invention, the level of one or more further atheroma markers is measured in a sample from the individual and it is assessed whether the level of the one or more further markers is indicative of atheroma in the individual (according to the first aspect of the invention) or whether the level of the one or more further markers is indicative of a particular outcome for the individual (according to the second aspect of the invention). Conveniently, the level of lysozyme and of the further atheroma marker are measured in the same sample taken from the individual. Alternatively, the levels may be measured in separate samples taken from the individual. Conveniently, the samples are taken from the individual at substantially the same time, or within several hours of each other.

Preferably, the level of the further atheroma marker is determined using a reagent that selectively identifies the further marker in a sample from the individual. Conveniently, the reagent binds to the further marker, and more conveniently the reagent is an antibody to the further marker. It will be appreciated that the antibody is typically one which has been raised to or selected using the further marker. Preferably, antibodies which bind preferentially to the further marker compared to other molecules in the sample taken from the individual (eg plasma) are used. Preferably, the antibody has at least a 10-fold higher affinity for the further marker than for any other component in the sample taken from the individual and more preferably at least a 50 fold, 100 fold, 500 fold, 1000 fold or 10000 fold-higher affinity.

Preferably, the further atheroma marker is any of cholesterol, lipocalin-type prostaglandin D, glycated albumin, myeloperoxidase or lipid peroxidase. The sample may be any suitable sample, but is typically blood, serum or plasma as defined above.

The method of the invention may be used to assist in the diagnosis and prognosis of atheroma, for example by aiding in distinguishing atheroma from other conditions which display similar symptoms, and thus lead to a more reliable diagnosis and prognosis of atheroma. Thus in a further embodiment, the methods of the first or second aspects of the invention further comprise performing any one or more of a coronary angiography (CAG), exercise stress testing (ETT), myocardial perfusion scanning, stress echocardiography, CT angiography using multi-detector row CT (MDCT), magnetic resonance imaging, positron emission tomography, high resolution computerised tomography scanning and Doppler ultrasound of arteries, on the individual to aid in the diagnosis or prognosis of atheroma in the individual.

In a preferred embodiment, the levels of lysozyme and of the one or more further atheroma markers are all taken into account when assessing whether the levels are indicative of atheroma in the individual or are indicative of a particular outcome for the individual. Thus, it will be appreciated that the assessment (eg diagnosis or prognosis) may be made on the basis of the level of lysozyme and the level of a further atheroma marker in a sample from the individual. Using a combination of markers may improve the accuracy of the assessment (eg diagnosis or prognosis), but nevertheless determining the level of lysozyme alone in a sample from the individual is useful.

A third aspect of the invention provides the use of a reagent which selectively identifies lysozyme in the assessment of atheroma in an individual. For example, the invention includes the use of a reagent which selectively identifies lysozyme in the diagnosis or prognosis of atheroma in an individual. The invention includes the use of a reagent which selectively identifies lysozyme in the assessment of whether an individual is suffering from atheroma. The invention also includes the use of a reagent which selectively identifies lysozyme in the assessment of the outcome for an individual who has atheroma. Suitable reagents are disclosed above, and it is particularly preferred to use an antibody to lysozyme in the diagnosis or prognosis of atheroma in an individual. Thus, the invention includes a reagent which selectively identifies lysozyme, such as an antibody, for use in assessing individuals with respect to atheroma as discussed above, eg for use in diagnosing or prognosing atheroma. The atheroma may be in any one or more of the aorta, a coronary artery, a renal artery, a neck artery, a cerebral artery, a limb artery or a visceral artery. Typically, the reagent is used in the methods described above. Preferably, the reagent selectively identifies lysozyme. Preferably, the individual is a human individual.

As discussed in more detail below, the reagent which identifies lysozyme may be used to assess whether a test compound has an effect on atheroma in an individual (typically a human individual) by altering the level of lysozyme in the individual.

In an embodiment, the third aspect of the invention also includes the use of a reagent that selectively identifies a further atheroma marker in the assessment of atheroma in an individual (eg diagnosis or prognosis). Suitable reagents that selectively identify the further atheroma marker are described above, but conveniently the reagent is an antibody. Thus, conveniently, the invention includes an antibody to lysozyme and an antibody to a further atheroma marker both for use in assessing atheroma in a patient eg diagnosing or prognosing atheroma in an individual. It is particularly preferred if the antibody to lysozyme and the antibody to the further atheroma marker are used in an immunoassay for assessing, eg diagnosing or prognosing, atheroma. The immunoassay may be for each marker individually, or it may be for two or more markers combined, for example a single immunoassay which is able to detect lysozyme and another atheroma marker. Preferably, the individual is a human.

The invention includes the use of a reagent which selectively identifies lysozyme in the manufacture of a composition for assessing atheroma in an individual.

The invention also includes the use of a reagent which selectively identifies lysozyme and the use of a reagent which selectively identifies a further atheroma marker in the manufacture of a composition for assessing atheroma in an individual.

The composition is a reagent which is used to assess atheroma in an individual. The composition manufactured may be used in the diagnosis of atheroma in an individual. The composition manufactured may be used in the prognosis of atheroma in an individual. The atheroma may be in any one or more of the aorta, a coronary artery, a renal artery, a neck artery, a cerebral artery, a limb artery or a visceral artery. Typically, the reagent which selectively identifies lysozyme and the reagent that selectively identifies a further atheroma marker are as described above. Preferably, the individual is a human.

The invention includes a reagent which selectively identifies lysozyme for use in assessing atheroma in an individual.

The invention also includes a reagent which selectively identifies lysozyme and a reagent that selectively identifies a further atheroma marker for use in assessing atheroma in an individual.

The reagent or reagents may be used in the diagnosis of atheroma in an individual. The reagent or reagents may be used in the prognosis of atheroma in an individual. The atheroma may be in any one or more of the aorta, a coronary artery, a renal artery, a neck artery, a cerebral artery, a limb artery or a visceral artery. Typically, the reagent which selectively identifies lysozyme and the reagent that selectively identifies a further atheroma marker are as described above. Preferably, the individual is a human.

A fourth aspect of the invention provides a kit of parts comprising a reagent that selectively identifies lysozyme and a reagent which identifies a further marker of atheroma. The atheroma may be in any one or more of the aorta, a coronary artery, a renal artery, a neck artery, a cerebral artery, a limb artery or a visceral artery. Conveniently, the reagents bind the lysozyme and the further atheroma marker; more conveniently, the reagent is an antibody as described above. It is particularly preferred if the further marker of heart failure is any of cholesterol, lipocalin-type prostaglandin D, glycated albumin, myeloperoxidase or lipid peroxidase.

In a particularly preferred embodiment, the kit is an immunoassay kit. In addition to the (primary) antibody to lysozyme and the (primary) antibody to the one or more further atheroma markers, the kit may also contain secondary antibodies to the primary antibodies. Conveniently, the primary antibodies are of different isotypes so that they can be distinguished by different secondary antibodies. Conveniently, the secondary antibodies are labelled differently so that they can be distinguished. In this way, it is possible for a single immunoassay to determine the level of lysozyme and one or more further atheroma markers in the same sample from the individual.

Another aspect of the invention provides the use of a means for selectively identifying lysozyme in a sample from an individual in the assessment of atheroma in the individual.

The means for selectively identifying lysozyme in the sample may be any suitable means. For example, in one embodiment the means is a mass spectrometer arranged to detect lysozyme in the sample. As noted above, lysozyme has a major peak at m/z 14735 and a minor peak at m/z 7375 when analysed by SELDI-TOF mass spectrometry.

The means may also be capable of selectively identifying a further atheroma marker in the sample.

The measurement of lysozyme in an individual (particularly a human individual) is also useful in assessing treatments of atheroma. Thus, a further aspect of the invention provides a method of assessing whether a compound has an effect on atheroma in an individual, the method comprising administering to the individual the said compound and determining the effect of the compound on the level of lysozyme in the individual. The atheroma may be in any one or more of the aorta, a coronary artery, a renal artery, a neck artery, a cerebral artery, a limb artery or a visceral artery. In this embodiment, the individual may be a laboratory animal such as a rat, mouse, guinea pig, dog or primate.

Methods for determining the level of lysozyme are described above. Typically, the level of lysozyme in the individual's blood, plasma or serum is determined.

In a further embodiment the level of a further atheroma marker, such as any of cholesterol, lipocalin-type prostaglandin D, glycated albumin, myeloperoxidase or lipid peroxidase is determined.

The method may be employed, for example, in the context of establishing whether a particular treatment is effective for a particular individual. Alternatively, the method may be employed, for example, in the context of a clinical trial of a candidate treatment, eg a drug, for atheroma. In this latter embodiment, the method is typically performed on a population of individuals. Thus, for example, the method may be carried out on at least 10, 50, 100, 200, 300, 400, 500 individuals, or at least 1000 individuals, or at least 5000 individuals or more.

Accordingly, the method of assessing whether a compound has an effect on atheroma, may comprise: determining the level of lysozyme in a population of individuals; administering the compound to the population of individuals; determining the level of lysozyme in a population of individuals subsequent to the administration step; and comparing the mean level of lysozyme determined before the administration step with the mean level of lysozyme determined subsequent to the administration step.

As is well known in the art, to control for the ‘placebo effect’, it may be desirable to substitute the compound for a placebo in a proportion of the individuals undergoing the clinical trial.

The compound may be administered as an individual dose or in several doses over a period of 1, 2, 3 or 4 weeks, 2, 4, 6, 6-12, 12-18 or 18-24 months, or several years, depending upon the compound and route of administration.

Typically, the level of lysozyme in the individual is determined immediately prior to the commencement of administering the compound.

The level of lysozyme in the individual subsequent to the administration step is typically determined about 2-4 weeks after the commencement of administering the compound. The level of lysozyme in the individual subsequent to the administration step may be determined multiple times, for example at regular intervals such as weekly, monthly, every six months or every year in order to monitor efficacy of the compound over time.

The inventors have demonstrated a correlation between lysozyme and atheroma. Thus, an alteration in the level of lysozyme subsequent to administration of the compound is an indication of the atheroma improving (ie lysozyme levels decrease) or advancing (ie lysozyme levels increase) since the compound was administered.

It is appreciated that the compound may affect the rate of progression of atheroma. Thus, it may be desirable to compare the change, or rate of change, in the level of lysozyme in an individual administered the compound to the corresponding change, or rate of change, in the level of lysozyme in an individual administered a placebo treatment. In this way, it is possible to ascertain if the atheroma has progressed, if its progression has slowed, its progression has stopped or its progression has been reversed. It may also be desirable to compare the effects of a compound with an alternative treatment for atheroma.

A fifth aspect of the invention provides a method of combating atheroma in an individual, the method comprising diagnosing atheroma in the individual according to the methods of the first aspect of the invention, and treating the atheroma.

By ‘combating’ we include the meaning that the invention can be used to alleviate symptoms of the disorder (ie palliative use), or to treat the disorder.

In one embodiment, combating the atheroma comprises administering at least one of a statin; a lipid lowering drug; a drug which modifies the angiotensin system such as an angiotensin converting enzyme inhibitor, an angiotensin receptor blocker or a rennin antagonist; an anti-platelet; an anti-coagulant drug; and a disease modifying agent for diabetes to the individual. For example, the disease modifying agent for diabetes may be any of insulin (eg insulin glargine), pioglitarcone (thiazolidinedione-type drug) or glimepiride (sulphonylurea-type drug). These agents may be used in combating, or used in the manufacture of a medicament for combating, atheroma in an individual who has been diagnosed as having atheroma according to the methods of the first aspect of invention.

For example, the invention includes the use of at least one of a statin; a lipid lowering drug; a drug which modifies the angiotensin system such as an angiotensin converting enzyme inhibitor, an angiotensin receptor blocker or a rennin antagonist; an anti-platelet; an anti-coagulant drug; and a disease modifying agent for diabetes, in combating atheroma in an individual who has been diagnosed as having atheroma according to the methods of the first aspect of the invention.

Similarly, the invention includes the use of at least one of a statin; a lipid lowering drug; a drug which modifies the angiotensin system such as an angiotensin converting enzyme inhibitor, an angiotensin receptor blocker or a rennin antagonist; an anti-platelet; an anti-coagulant drug; and a disease modifying agent for diabetes, in the manufacture of a medicament for combating atheroma in an individual who has been diagnosed as having atheroma according to the methods of the first aspect of the invention.

The invention also encompasses a method of combating atheroma, the method comprising diagnosing atheroma in the individual according to the methods of the first aspect of the invention, and treating the atheroma, for example by the application of a treatment regime based upon diet, exercise or other lifestyle change, or pharmaceutical intervention, or a combination thereof.

The atheroma may be any one or more of the aorta, a coronary artery, a renal artery, a neck artery, a cerebral artery, a limb artery or a visceral artery. Preferences for the individual are given above with respect to the first aspect of the invention. Preferably, the individual is a human individual.

All documents cited in the patent specification are hereby incorporated herein by reference.

The invention will now be described in more detail by reference to the following non-limiting Examples and Figures wherein:

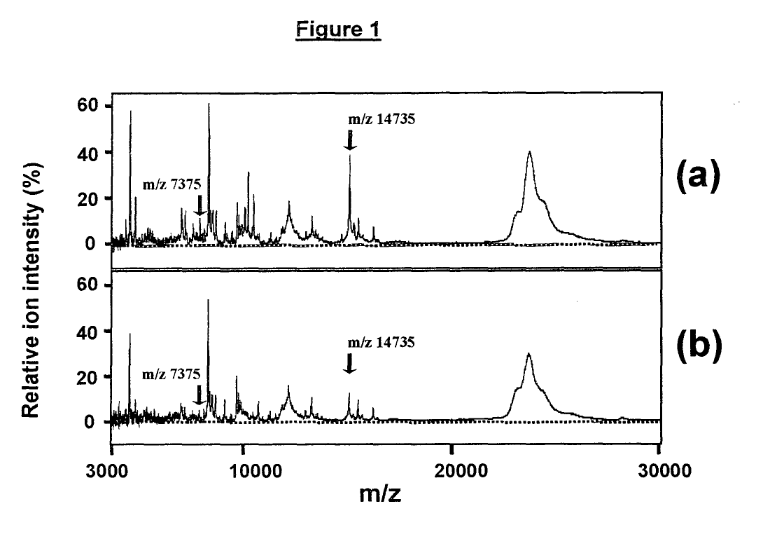

FIG. 1—Representative SELDI-TOF MS spectra from the plasma of 3VD (a) and NV (b). Arterial plasma (15 μl) was diluted in 1:10 ratio in 9 M Urea, 2% CHAPS and 5 mM DTT followed by a 1:10 dilution in binding buffer (0.1 M sodium acetate; 0.1% triton X-100; pH6) and loaded on a CM10 ProteinChip array. The array was washed three times with binding buffer followed by a quick wash with deionised water. SPA (0.5 μl) was applied twice onto the dried spots and data was acquired at laser intensity 205. Two peaks at m/z 14735 and its doubly charged m/z 7375 (indicated by an arrow) were found to discriminate the 3VD from the NV group. The baseline of the spectra is represented by dotted lines.

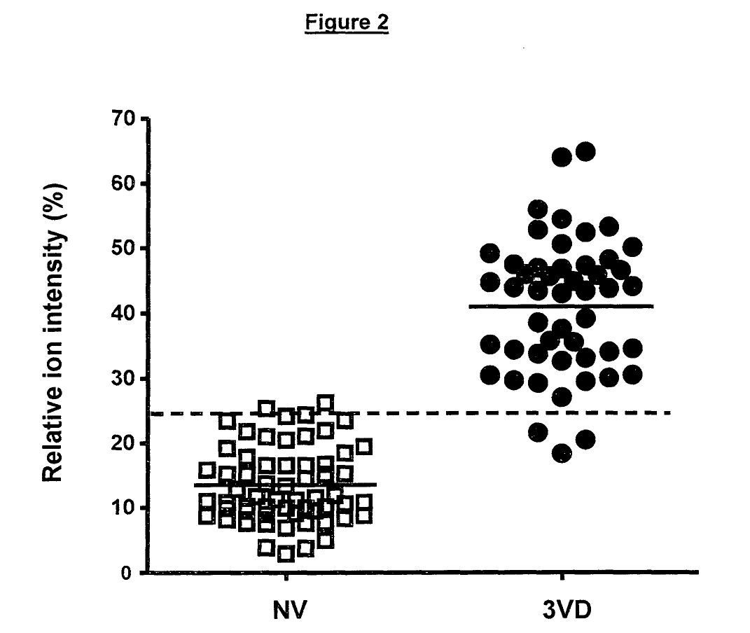

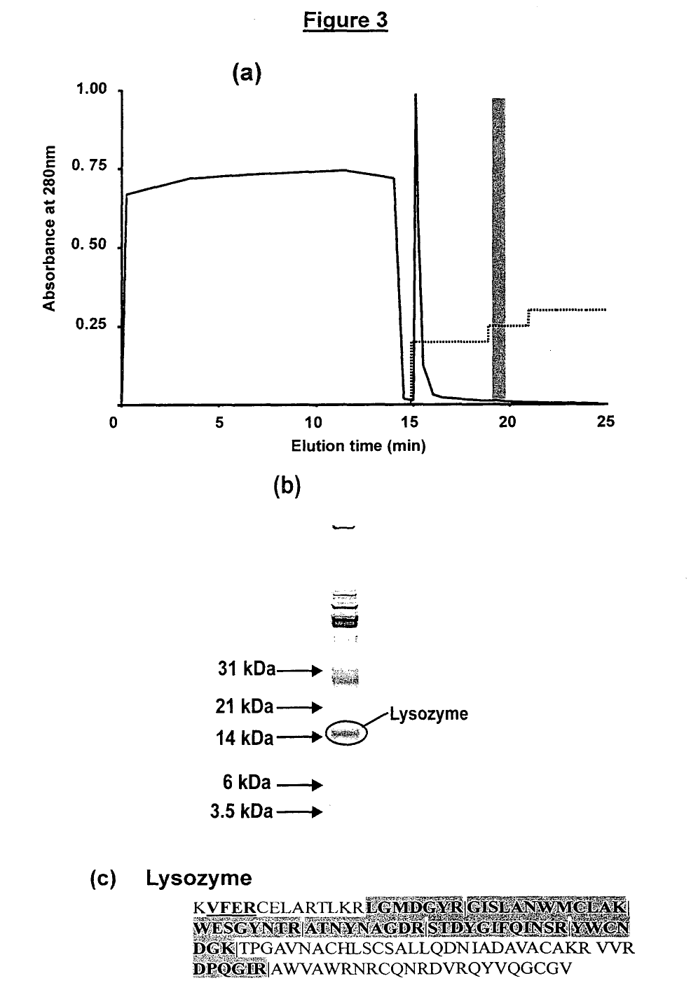

FIG. 2—Distribution of m/z 14735 ion in 3VD and NV plasma samples. 3VD (n=42) and NV (n=52) samples were processed and analysed as described in Example 1. Data are presented as the average point of replicates of peaks in each sample with their means indicated by a solid line. A cut off at the relative intensity value of 25 (dotted line) gave 90% specificity and 95% sensitivity.

FIG. 3—Purification and identification of M_{14.7}. Whole plasma was fractionated by (a) ion exchange chromatography using a CM Hyper DF column equilibrated in 0.1 M sodium acetate, pH 6 and eluted with a stepwise gradient of increasing NaCl concentrations. Fractions were collected and analysed on CM10 ProteinChip arrays and those that contained M_{14.7} (shaded block) were pooled, concentrated using Vivaspin 20 tubes and (b) separated by SDS-PAGE. The band corresponding most closely to a mass of 15 kDa was excised and subjected to trypsin digestion and peptide analysis by MS. (c) Seven peptides were identified (highlighted in grey) that originated from human lysozyme. N-terminal sequencing of the protein found in the band (underlined) also corresponded to human lysozyme.

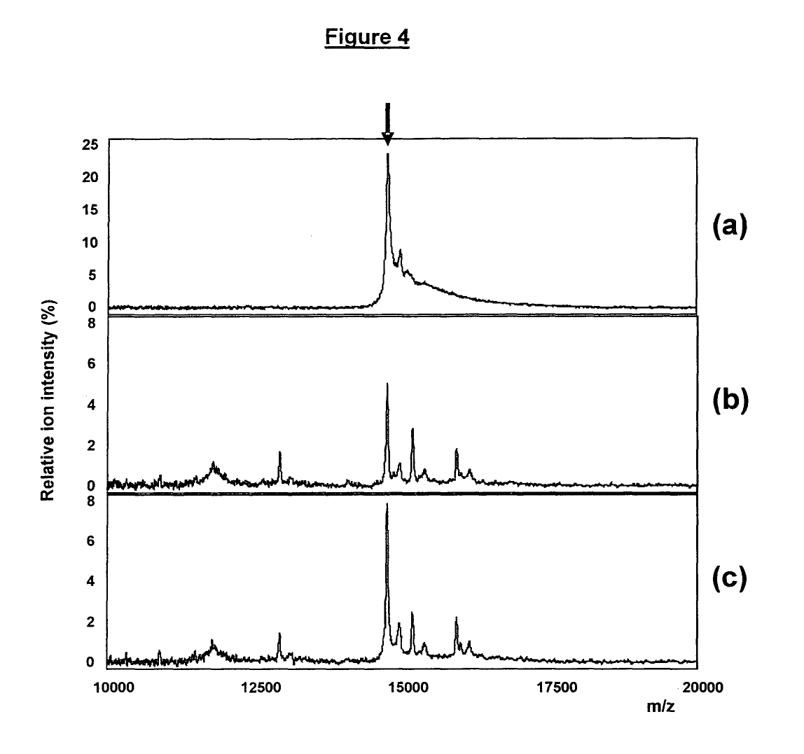

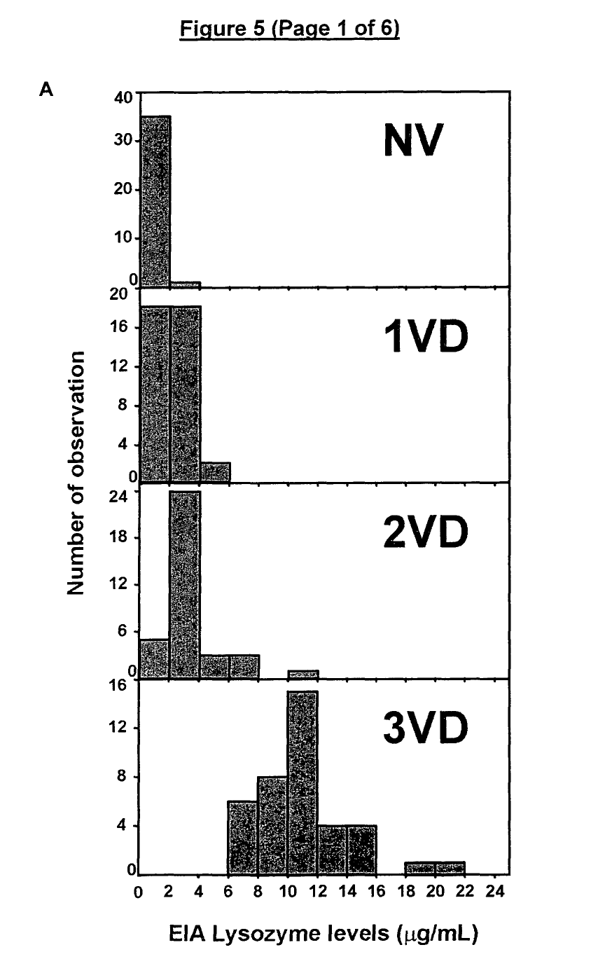

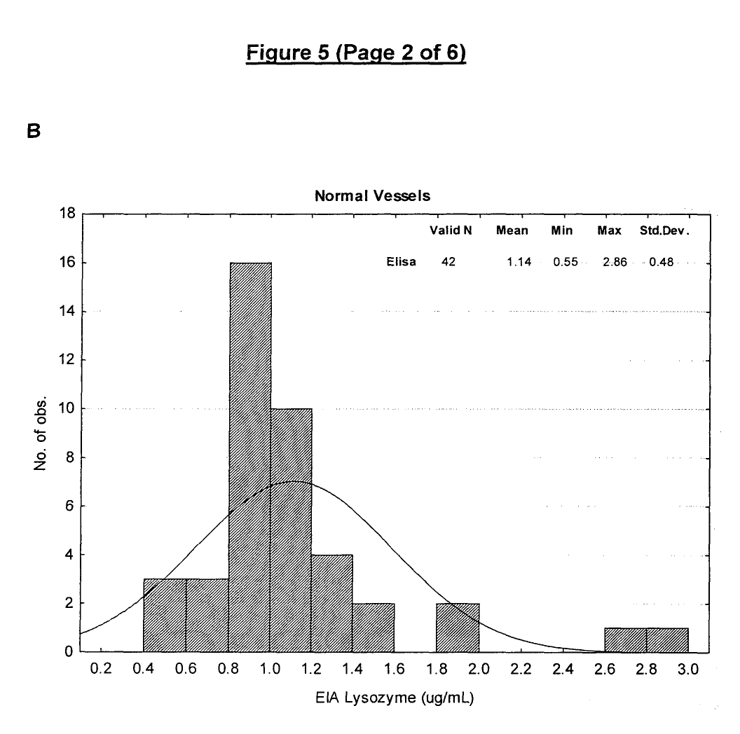

FIG. 4—Confirmation of the identity of M_{14.7} as lysozyme. Samples of (a) authentic human lysozyme (10 ng), (b) plasma (15 μL) from an NV patient, and (c) a mixture of lysozyme and NV plasma were applied to CM10 ProteinChip arrays at pH 6 and analysed under the conditions described in FIG. 1. The positions of m/z 14735 ion the spectra due to lysozyme in the authentic protein as in the plasma sample are indicated by arrows.

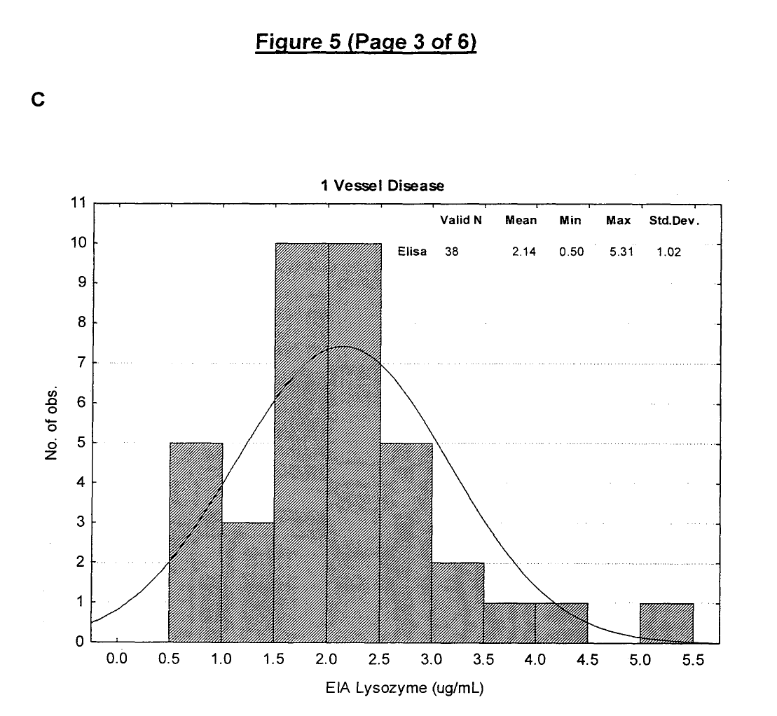

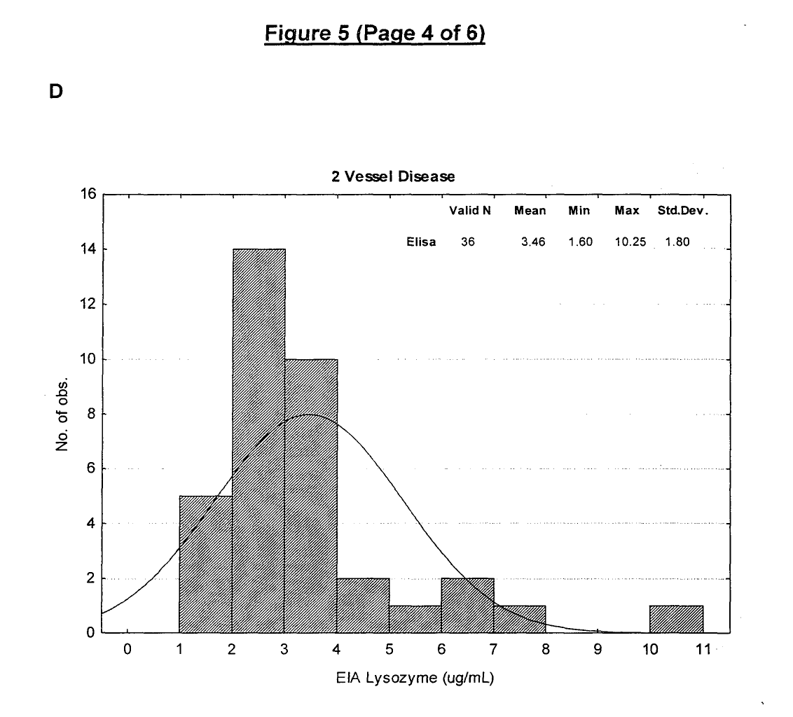

FIG. 5—Distribution of plasma lysozyme in normal and diseased vessels. (A) Distribution of lysozyme levels in the arterial plasma of patients with NV (n=42), 1VD (n=38), 2VD (n=36) and 3VD (n=46) were determined using an EIA. Samples were grouped in an interval of 2 μg/ml and in each case, the mid-point of the group is shown. Plasma lysozyme levels were significantly elevated in each of the groups of patients with evidence of CAD (1VD, 2VD, 3VD) compared to the NV group (p<0.0001, Student's t-test). A one way ANOVA analysis also showed significant differences amongst the groups (p<0.001) with a r² value of 0.83. This same data is also prevented as a series of histograms that show the distribution of arterial plasma lysozyme levels along with Gaussian distribution curves based on the assumption of a normal distribution of data in each case for (B) NV, (C) 1VD, (D) 2VD, (E) 3VD, and (F) data for all 4 groups combined.

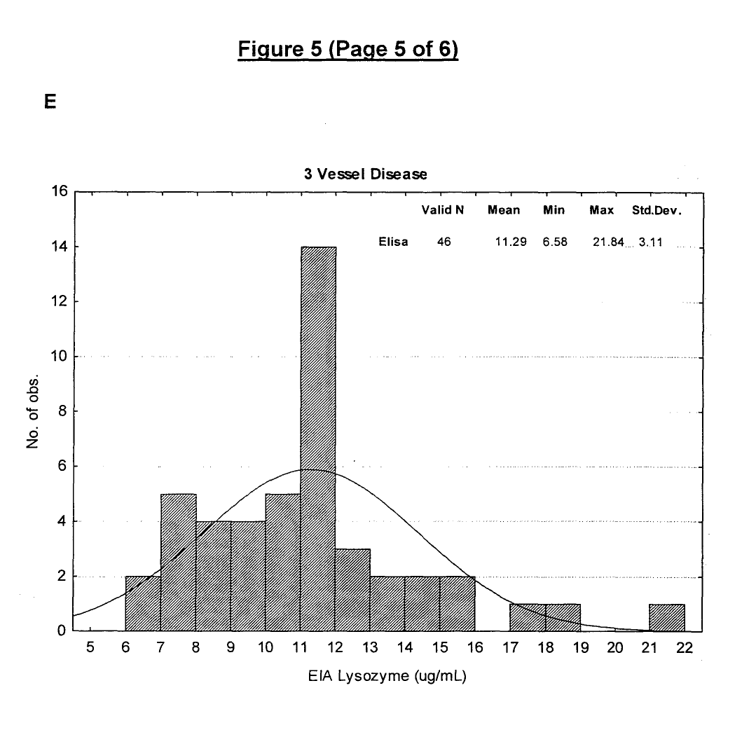

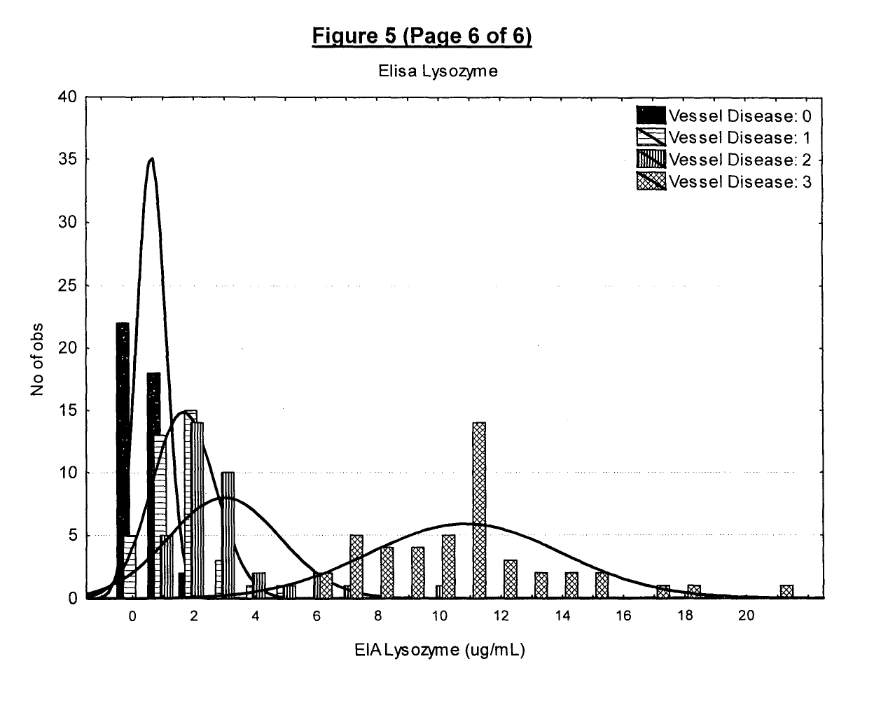

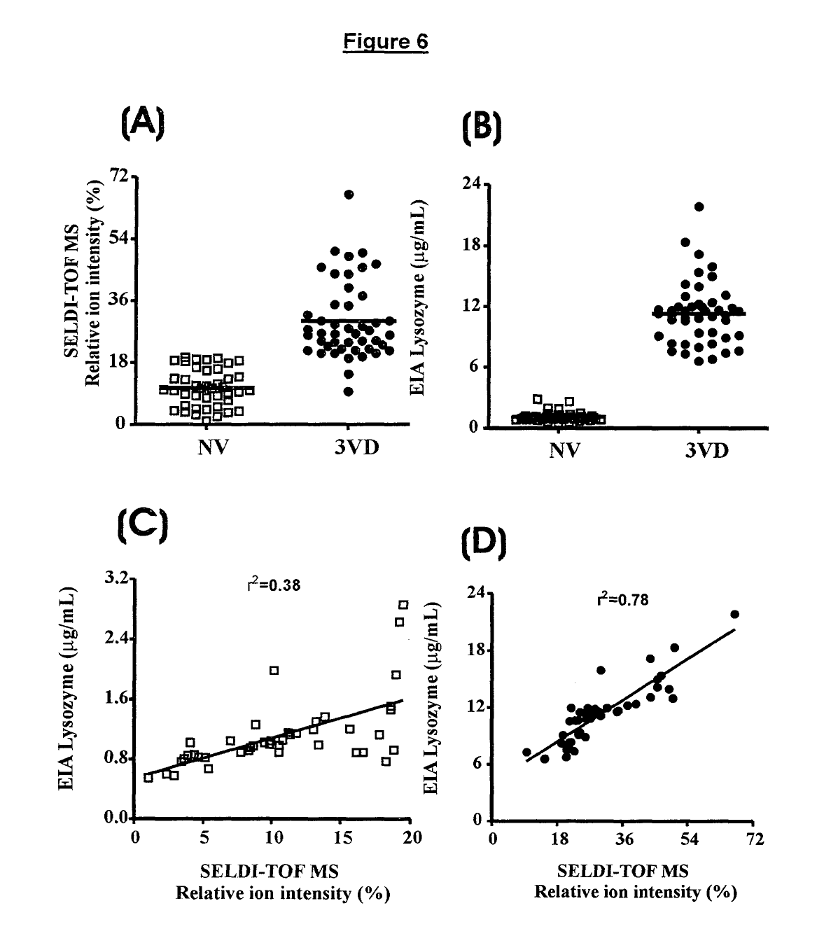

FIG. 6—Relationship between the levels of lysozyme determined by SELDI-TOF MS and EIA. (A) The ion intensities of M_{14.7} from SELDI-TOF MS analysis was compared to (B) EIA lysozyme measurements in the same group of samples of 42 NV and 46 3VD patients. In both methods, the 3VD group had significantly elevated levels of M_{14.7} or lysozyme compared to NV (p<0.0001, Student's t-test). The relationship between the ion intensities of M14.7 from SELDI-TOF MS analysis to the EIA lysozyme determination were significant (p<0.0001) in both the (C) NV and (D) 3VD group, with the latter displaying high correlation values.

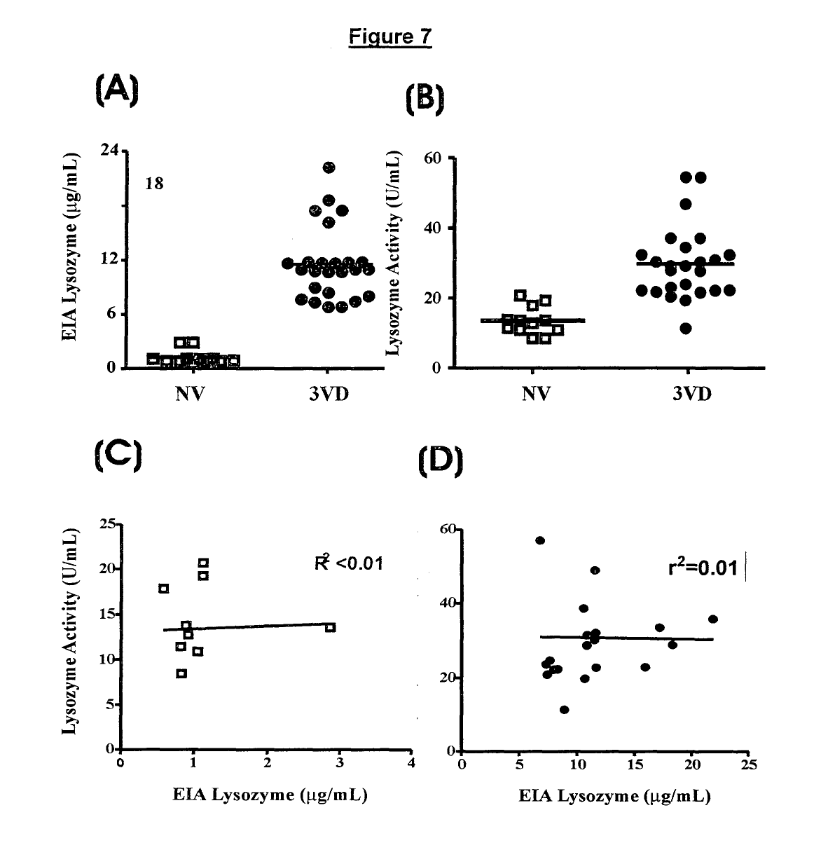

FIG. 7—Activity of lysozyme in NV versus 3VD and its relation to EIA Lysozyme. (A) Plasma lysozyme levels determined in 12 NV and 25 3VD patients using an EIA showed significant elevation in 3VD compared to NV (p<0.0001, Student's t-test) with no overlaps between the two groups. (B) The measurement of lysozyme activity of the same group of samples using total activity assay also revealed significant elevation in 3VD compared to NV (p<0.0001, Student's t-test) with a considerable overlap between the measurements from each group. There was no relationship (r²<0.01) between the levels of EIA lysozyme and lysozyme activity in all the samples in either the NV group (C) or the 3VD group (D). The average ratio of Lysozyme activity to EIA levels in each samples were 15.0 in NV and 2.8 in 3VD, indicating markedly reduced amount of active lysozyme in 3VD patients.

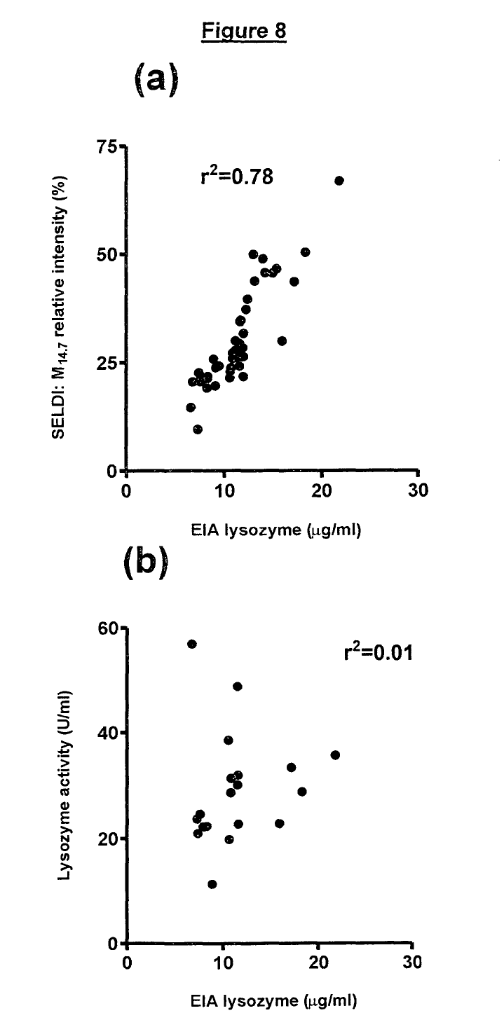

FIG. 8—Relationships between arterial plasma lysozyme levels determined by EIA, SELDI-TOF MS, and enzyme activity assays. Arterial plasma lysozyme levels in patients with 3VD CAD were measured by EIA and compared with determinations by (a) SELDI-TOF MS measuring the ion intensity of M_{14.7} (n=46) and (b) enzyme activity of lysozyme (n=25). Whilst there is a close association between the levels determined by EIA and SELDI-TOF MS, enzyme activity measurements are unrelated.

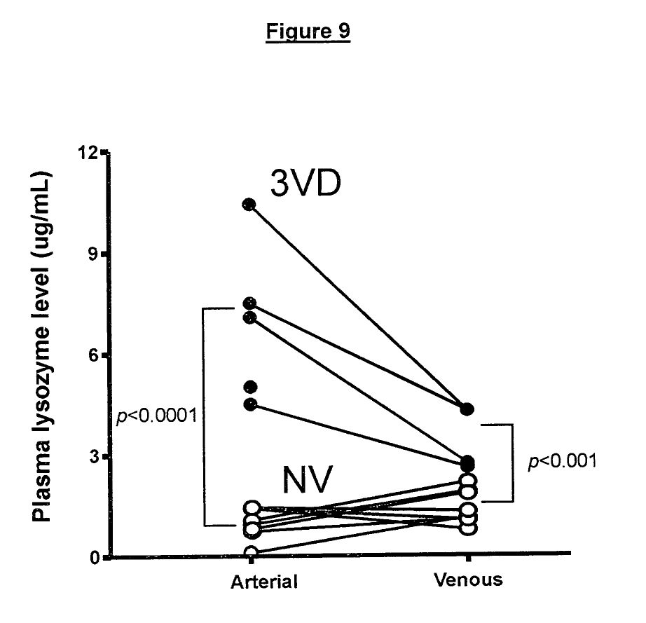

FIG. 9—Correlation of arterial and venous plasma lysozyme level with coronary artery disease. Lysozyme levels in arterial and venous plasma determined in 4 3VD and 8 NV patients using an EIA showed significant elevation in 3VD compared to NV patients.

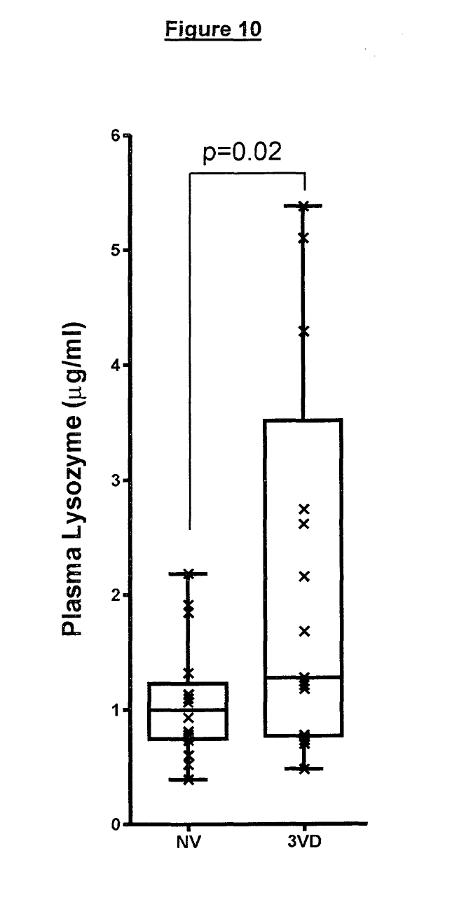

FIG. 10—Venous plasma lysozyme in patients with atheroma. Venous plasma lysozyme levels were determined in individuals that appeared free of atherosclerosis by angiography with normal coronary arteries (NV) (n=16) and compared with those with 3 vessel disease (3VD) (n=17). The data in each group is summarised by a box and whisker plot that indicates in each case values corresponding to the median, 25% and 75% quartile and range. A statistically significant increase in lysozyme levels in patients with 3VD was apparent (p<0.02, Student's t-test).

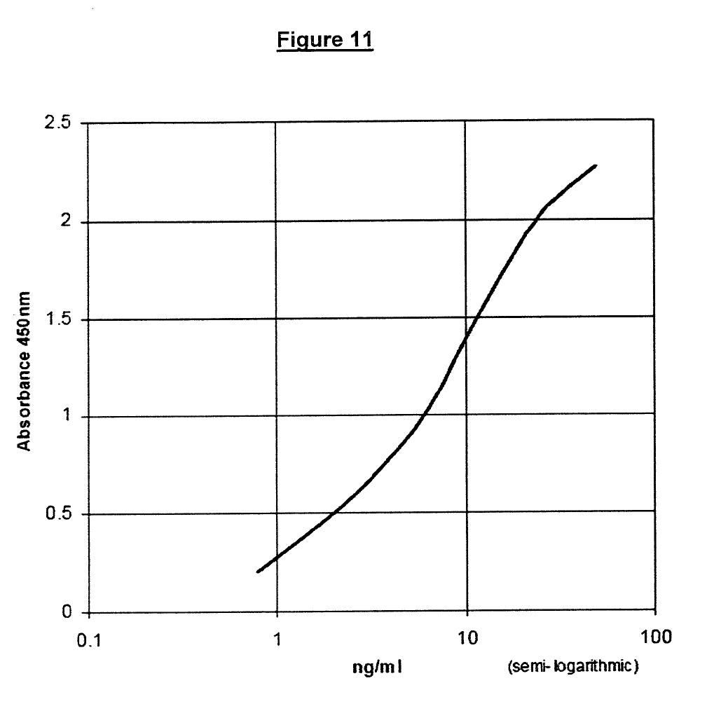

FIG. 11—Typical standard curve generated using Biomedical Technologies Inc Human Lysozyme EIA kit

### EXAMPLE 1

### Identification of Arterial Plasma Lysozyme as a Biomarker of Coronary Artery Disease

### Summary

An estimated 80 million American adults (1 in 3) have 1 or more types of cardiovascular disease (CVD) secondary to atherosclerosis (2). The early detection and accurate assessment of vascular atheromatous plaques permits timely intervention with lifestyle changes and pharmacological agents. Currently, the detection and assessment of atheroma is dependent upon functional tests and/or access to imaging. For example, the evaluation of patients with suspected coronary artery disease (CAD) comprises exercise stress testing (EU), followed by coronary angiography (61). However, meta-analyses have shown that EU has a sensitivity and specificity of only 65-70% and 70-75% respectively (6, 7). Around 15-20% of patients referred for coronary angiography following EU have no sign of atheromatous coronary disease (8). Other non-invasive methods of diagnosis such as myocardial perfusion scanning and stress echocardiography fare better (sensitivity 75-85%, specificity 85-90%) (9). Computed tomography (CT) coronary angiography using multi-detector row CT (MDCT) appears to have the best diagnostic accuracy (sensitivity 85-90%, specificity 85-95%) (10, 11), although this varies with patient and lesion characteristics (10, 12). Carotid atheroma can be detected by Doppler ultrasound but more sophisticated techniques, still under investigation, are required to assess plaque vulnerability. A soluble biomarker of vascular atheroma would have considerable clinical utility.

### Materials & Methods

### Materials

ProteinChip arrays (CM10), all-in-one peptide standard, all-in-one protein-standard and cation exchange chromatography sorbent media (CM HYPER® DF) were from Ciphergen Biosystems Inc. (Freemont, Calif., USA). Sinapinic acid (SPA) and alpha-cyano-4-hydroxy cinnamic acid (CHCA) (>99% purity for MALDI-MS) were from Fluka/Sigma-Aldrich Company Ltd. (Poole, UK). HPLC grade acetonitrile, formic acid and methanol were obtained from VWR International Ltd. (Lutterworth, UK). Trifluoro acetic acid (TFA) was from Rathburn Chemicals Ltd. (Walkersbum, UK). Precast Nupage® Novex 10% bis-tris gels, SeeBlue® pre-stained molecular markers, Novex® 2-(N-morpholino)ethane sulphonic acid (MES) SDS running buffer, lithium dodecyl sulphate (LDS) sample buffer and Nupage® antioxidant were from Invitrogen Ltd. (Paisley, UK). Vivaspin concentrators (20 ml, 3 kDa molecular weight cut off) were from Vivascience AG (Hannover, Germany). Human lysozyme enzyme immuno assay kits (EIA) containing lysozyme standard (2 μg/mL) were purchased from ODS Ltd (Tyne and Wear, UK). InstantBlue® was obtained from Novexin Ltd (Cambridgeshire, UK) and sequencing grade modified trypsin was from Promega UK (Southampton, UK). All other reagents were purchased from Sigma-Aldrich Company Ltd. (Dorset, UK).

### Patients and Plasma Sample Collection

samples of arterial blood were collected from 201 patients who attended the cardiac Catheter Laboratories at Wycombe Hospital for coronary angiography. All patients had a clinical diagnosis of angina based on a combination of history, physical examination, risk factors and non-invasive investigations such as an exercise tolerance test. The results were sufficient to warrant a diagnostic angiography to investigate further. Local ethical approval was obtained for this study. Details of patient demographics are presented in Table 1. The severity of coronary artery disease was quantified from the angiogram as the number of arteries where the vessel lumen was occluded by more than 60%. Patients were divided into four groups: no disease (normal vessel; NV), 1 affected vessel (1 vessel disease; 1VD), 2 affected vessels (2 vessel disease; 2VD) and 3 affected vessels (3 vessel disease; 3VD), respectively. Blood was collected from a femoral or radial artery through the catheter used in the angiography procedure. Blood samples were collected prior to the administration of any drugs or other reagents and before the procedure proper had commenced. A 5 mL sample of blood was collected in a citrate tube, which was mixed and then placed on ice. In some cases, venous blood samples were collected from the median cubital vein into citrate tubes. Plasma was separated by centrifugation (950×g for 10 min) within 30 min of collection and then stored frozen at −20° C. for up to 1 month. Following transport to the laboratory, plasma samples were thawed on ice, divided into 200 μL volumes, and stored frozen at −80° C. until analyses were performed.

TABLE 1

| | (n = 60) | (n = 49) | (n = 42) | (n = 50) |
|---|---|---|---|---|
| Baseline patient | | | | |
| characteristics | | | | |
| Age in years | 58.2 ± 0.5 | 66.4 ± 1.6 | 67.7 ± 1.6 | 70.0 ± 01.5 |
| (Mean ± SEM) | | | | |
| Gender (male/female) | 25%/75% | 63%/37% | 74%/26% | 64%/36% |
| Current smoker | 7% | 4% | 7% | 6% |
| BMI in kg/m² | 28.2 ± 0.7 | 27.5 ± 0.8 | 26.2 ± 0.5 | 27.4 ± 0.6 |
| (Mean ± SEM) | | | | |
| TC/HDL | 3.8 ± 0.2 | 4.2 ± 0.2 | 3.9 ± 0.2 | 3.6 ± 0.2 |
| (Mean ± SEM) | | | | |
| Concomitant/ | | | | |
| Past Medical History | | | | |
| Diabetes | 10% | 6% | 21% | 24% |
| Hypertension | 29% | 37% | 48% | 58% |
| Hypercholesterolaemia | 47% | 57% | 67% | 72% |
| Previous MI | 2% | 6% | 12% | 18% |

### SELDI-TOF MS, Biomarker Purification and Identification

Plasma samples were processed and analysed by SELDI-TOF MS as previously described (24). A preliminary study was conducted to determine appropriate ProteinChip array (Bio-Rad Laboratories Ltd., Hemel Hempstead, UK) conditions to use for sample analysis. The final conditions established to analyse the plasma samples were dilution of plasma 1:10 in 9 M urea, 2% 3-[(3-cholamidopropyl)dimethylammonio]-1-propanesulphonate, 5 mM dithiothreitol, which was then diluted 1:10 in 0.1 M sodium acetate with 0.1% triton X-100 at pH 6 prior to application to CM10 ProteinChip arrays. A saturated solution of 1 μL sinapinic acid (0.5 μl applied twice) in 50% (v/v) acetonitrile and 0.5% (v/v) trifluoroacetic acid was applied to each spot followed by analysis on Protein Biology System IIc Reader (Bio-Rad Laboratories Ltd., Hemel Hempstead, UK). All samples were analysed in duplicate.

### Purification and Protein Identification

The purification method of the selected biomarker followed previously described methods (24). Ion exchange fractionation was performed on a CM Hyper DF column (BioSepra Inc., Massachusetts, USA). Fractions containing the candidate biomarker were pooled, lyophilised and separated by sodium dodecyl sulphate-polyacrylamide gel electrophoresis (SDS-PAGE) using Nupage 10% bis-tris gels (Invitrogen Ltd., Paisley, UK), which were stained with InstantBlue Coomassie stain (Novexin Ltd., Cambridge, UK). The identity of the protein in the band was determined by peptide mass fingerprinting following trypsin digestion overnight and also by N-terminal sequencing of the intact protein. Tryptic peptides were extracted and analysed by liquid chromatography (LC) tandem MS using an Ettan MDLC (GE Healthcare Inc., Little Chalfont, UK) interfaced with a Linear Ion Trap MS (Thermo Fisher Scientific, Inc. Hemel Hempstead, UK). The identity of the peptides and protein(s) were determined using the SEQUEST search engine (BioWorks Browser 3.3; Thermo Fisher Scientific, Inc. Hemel Hempstead, UK) to interrogate the RefSeq human database (downloaded from the NCBI ftp website http://www.ncbi.nlm.nih.gov/RefSeq/). N-terminal sequencing of the excised protein was performed by Alta Bioscience, University of Birmingham (Birmingham, UK) using a Procise Protein Sequencer (model 494HT).

### Measurement of Lysozyme Levels and Activity

The levels of human lysozyme were measured using an enzyme immunoassay (EIA) kit (Biomedical Technologies Inc, Stoughton, USA). Lysozyme activity measurements were determined using EnzChek® Lysozyme Assay Kit (Invitrogen Ltd.; Paisley, UK) (Mod Pathol 7, 771 (1994); Acta Pathol Jpn 28, 689 (1978); N Engl J Med 277, 10 (1967); J Clin Pathol 36, 1312 (1983); Nephron 63, 423 (1993); Clin Chem 32, 1807 (1986); and Toxicology 28, 347 (1983)). In both cases the respective manufacturers' recommended protocols were used. Plasma samples were diluted in phosphate-buffered saline by 1:1000 for the EIA and 1:500 for activity measurements.

### Statistical Analyses

All statistical analyses were performed with STATISTICA 6.1 (Statsoft Inc, Tulsa, USA) and GraphPad Prism software version 2.0 (GraphPad software, San Diego, USA). Individual t-tests were performed to compare each variable in NV and 3VD groups and those with significant differences (p<0.05; Student's t-test) were selected for further analysis. Differences between levels were determined by analysis of variance (ANOVA) and Student's t-test, where appropriate.

### Results

### Expression Difference Analysis

SELDI-TOF MS plasma protein profiles from 60 patients with NV and 49 with 3VD were generated on CM10 ProteinChip arrays at pH 6. A quality control sample derived from pooled plasma of healthy individuals was included in all analyses. The coefficient of variation of the SELDI-TOF MS assay determined from 10 peaks detected in the quality control sample (16 repeat analyses) was 19.8%. Overall, a total of 145 protein ions were detected under these conditions and those that varied significantly (p<0.01; Student's t-test) in their intensity between the two groups were selected for further analysis. This reduced the data set to 2 protein ions i.e. m/z 7375 and m/z 14735 which were higher in the 3VD group compared to the NV group. The ion with m/z 7375 was a doubly charged from of m/z 14735 (FIG. 1). The analysis on the intensities of m/z 14735 between the two groups showed 95% specificity and 90% sensitivity (FIG. 2). Hence, the protein from which the m/z 14735 ion was derived (M_{14.7}) was chosen for further study.

### Purification and Identification of M_{14.7}

Pooled plasma (6 ml) from 12 patients was applied to a cation exchange column equilibrated in 0.1 M sodium acetate, pH 6 and bound proteins eluted with a gradient of increasing concentrations of sodium chloride. Fractions were tested for the present of M_{14.7} by SELDI-TOF MS. M_{14.7} was eluted from the column with 250 mM sodium chloride. Other components present in this fraction and were successfully separated by SDS-PAGE. The band migrating with an apparent mass equivalent to 14.7 kDa was excised, digested with trypsin and analysed by tandem MS. Altogether, 7 tryptic peptides were identified that matched to human lysozyme based on a high probability scores. This is equivalent to 41% of the coverage of the protein. The result was confirmed on two separate occasions. The identity of lysozyme was also determined by N-terminal sequencing. This showed that the N-terminus was XVFER (SEQ ID No: 2) which corresponds to the N-terminus of human lysozyme (FIG. 3). The terminal residue (X) could not be identified by this method, possibly as a result of a post-translational modification.

To further confirm this identification, SELDI-TOF MS analysis was carried out on a preparation of authentic human lysozyme. This produced a major peak at m/z 14735 that matched the size and shape of the m/z 14735 ion in plasma (FIG. 4a). When a plasma sample that contained a readily detected quantity of the m/z 14735 ion was spiked with authentic lysozyme, the intensity of the ion increased without altering the intensity of other protein ions in the spectrum (FIG. 4c), except an increase in m/z 7375 (which appeared to be the doubly charged ion of M_{14.7}). The levels of lysozyme measured using EIA correlated well with SELDI-TOF MS measurements, especially in the 3VD group, further confirming the identity of M_{14.7} (FIGS. 6d and 8a).

### Determination of Plasma Lysozyme Levels

Arterial plasma lysozyme levels were measured by EIA in all samples collected from patients undergoing angiography. The assay was performed blind. Once complete the values obtained were compared with the findings at angiography, which was used as a surrogate measure of the extent of atherosclerosis. There was a significant difference in the levels between the groups (p<0.0001, one way ANOVA) with a significant increase in lysozyme level with the severity of disease. NV patients had a mean level of 1.14±0.07 μg/mL (n=42). The level was raised 2-fold in those with 1VD (2.15±0.17 μg/mL; n=38, p<0.0001, Student's t-test), by 3-fold in those with 2VD (3.46±0.30 μg/mL; n=36, p<0.0001, Student's t-test) and by 11-fold in those with 3VD (11.29±0.46 μg/mL; n=46, p<0.0001, Student's t-test) (FIG. 5).

There was some variation in the composition of the patient groups, which required further consideration to eliminate them as confounding factors. Therefore, lysozyme levels in various subsets of the patients categorised as NV or one of the VD groups were also compared. This was done for age (stratified into decades), males/females, diabetics/non-diabetics, patients with/without a history of ishaemic heart disease, those on drug treatments (such as angiotensin-converting enzyme inhibitors, beta-blockers, statins, anti-platelets, and nitrates) and the artery from which the sample was obtained (radial or femoral). In all cases, lysozyme levels were significantly different (p<0.0001, one way ANOVA) in patients with VD compared to NV. Also, within each CAD group the comparative lysozyme levels between each of the demographic subsets was not significantly different (p>0.05, Student's t-test). There was also no correlation between plasma lysozyme levels with any of the traditional CVD risk factors such as body mass index, blood levels of low density lipoprotein, high density lipoprotein, triglyceride, total cholesterol and diastolic/systolic blood pressure, which indicates that lysozyme is an independent marker of the disease status.

Lysozyme performed particularly well in differentiating 3VD from NV with 100% sensitivity and specificity (FIG. 5). ROC curve analyses showed that the area under the curve (AUC) of plasma lysozyme levels in NV versus disease vessels was 0.95; with a sensitivity of 91% and specificity of 94% at a cut of value of 1.50 μg/mL. Although there is overlap between NV and 1VD and 2VD groups, levels can be set that distinguish these affected patients with 91% sensitivity and 80-100% specificity (Table 2).

TABLE 2
Receiver Operator (ROC) analyses of lysozyme levels in patients with
normal vessels and various degrees of affected coronary arteries

| | ROC | | Cut-off value | Sensitivity | Specificity |
|---|---|---|---|---|---|
| | AUC | p-value | (μg/mL) | (%) | (%) |
| NV vs all VD | 0.94 | <0.0001 | 1.5 | 91 | 94 |
| NV vs 1VD | 0.83 | <0.0001 | 1.5 | 91 | 80 |
| NV vs 2VD | 0.98 | <0.0001 | 1.6 | 91 | 100 |
| NV vs 3VD | 1 | <0.0001 | 5.0 | 100 | 100 |
| 1VD vs 2VD | 0.76 | 0.0003 | 2.5 | 73 | 65 |
| 2VD vs 3VD | 0.99 | <0.0001 | 6.5 | 92 | 98 |

### Plasma Lysozyme Activity Measurements

The activity of lysozyme was measured in arterial plasma samples from 12 patients with NV and 25 patients with 3VD. The NV group had a mean activity of 13.5±1.1 U/mL. Activity in the 3VD group was significantly raised by two-fold (30.8±2.2 U/mL; p<0.0001, Student's t-test). There was also a very poor correlation (r²=0.01) between the lysozyme activity and its levels in the plasma (FIGS. 7d and 8b). In terms of specific activity (i.e. the ratio of activity to quantity), lysozyme was clearly more active in the NV group (15.0±2.4 U/μg) compared with the 3VD group (2.8±0.4 U/μg) (p=0.0003, Student's t-test). This indicates that the majority of the lysozyme detected in the plasma of patients with 3VD by EIA was partially or completely inactive.

### Venous Plasma Lysozyme Levels

Arterial and venous plasma were simultaneously sampled from four patients with 3VD CAD and eight patients with no disease to determine if venous plasma lysozyme is also a biomarker of CAD. Plasma lysozyme levels were determined as described above. The results are illustrated in FIG. 9.

Although lysozyme levels were not as elevated in venous plasma as those in the corresponding arterial plasma samples, venous levels were nevertheless raised.

In a further study, venous levels were measured in patients with CAD and carotid atherosclerosis (FIG. 10). Subjects with no evidence of coronary atherosclerosis had a mean level of 1.08±0.13 μg/ml (n=16). The level in patients with 3VD was significantly higher; 2.15±0.40 μg/ml (n=17); p=0.02, Student's t-test. Samples from patients with carotid atherosclerosis also had significantly raised levels of 1.56±0.13 μg/ml (n=34) compared to patients without coronary artery disease; p=0.02, Student's t-test.

Thus, levels of lysozyme in venous blood may be used as a biomarker of coronary artery disease.

### Discussion

This study identified lysozyme in an unbiased screen of circulating proteins in arterial plasma from patients with symptomatic coronary atheroma, verified by coronary angiography, as a potential accessible biomarker of active vascular disease. It has biological plausibility, being present in the plaque itself, and levels correlate with the severity of coronary atheroma.

The performance of lysozyme as a putative clinical biomarker with 91% sensitivity and 94% specificity with an AUC of ROC analysis of 0.94 compares favourably with other putative biomarkers of atherosclerosis. Elevated plasma or serum levels of lipoprotein-associated phospholipase A2 (56), C-reactive protein (57), CD40 ligand (58), myeloperoxidase (58), lipocalin-type prostaglandin D (28), glycated albumin (29), F11 receptor/junctional adhesion molecule (59), osteoprotegerin (60) and various chemokines and cytokines (61) have been reported and some of these appear to associate closely with risk of future events. However, where stated, diagnostic performance values of sensitivity and specificity were relatively poor, as were analyses by AUC of ROC curves. The diagnostic accuracy of arterial lysozyme is similar to the most accurate non-invasive diagnostic test available, multi-dimensional CT, which has a sensitivity of 85-90% and specificity 85-95% (8). Notably, arterial plasma lysozyme levels were able to distinguish completely patients with 3VD from those with NV; such information may aid the prioritization of patients for further investigation.

In terms of clinical utility, venous blood is more readily and routinely sampled compared to arterial blood. Venous plasma lysozyme levels were similar to arterial levels in subjects with no evidence of atherosclerosis and were elevated in patients with CAD, although less so than those found in arterial plasma. Nevertheless, raised venous plasma levels were also found in patients with carotid artery disease.

The source of lysozyme in arterial and venous blood merits some discussion. Lysozyme is normally present in circulating blood monocytes and free plasma levels come from disintegrating neutrophilic granulocytes (31). Nevertheless, immunohistochemistry shows that lysozyme is also present in human atheromatous plaques (32, 33). The higher circulating level in arterial versus venous blood supports an arterial source of lysozyme in plasma. The correlation between levels and severity of arterial atheroma is consistent with the hypothesis that active plaque is a source of circulating lysozyme. These observations lend biological plausibility to the use of circulating lysozyme levels as a biomarker of active atheromatous disease.

Interestingly, circulating lysozyme levels do not correlate with lysozyme activity. Arterial plasma from CAD patients comprises predominantly partially or completely inactive lysozyme, which appears to originate from atheromatous plaques. Naito et al have reported that, unlike circulating monocytes which contain active lysozyme, lysozyme in lipid laden macrophages in plaques from cholesterol-fed rabbits is inactive (34). These data provide further support for the contention that a significant component of the lysozyme circulating in plasma originates from atheromatous plaques. Inactivation of lysozyme activity in the plaque is likely to be due to oxidative damage by myeloperoxidase, which has been shown to form hypochlorus acid that can react with lysozyme causing loss of enzyme activity (35, 36). Myeloperoxidase activity has been shown to co-localize with foamy macrophages in human atherosclerotic tissue where lysozyme is also found (32, 37). Thus, lysozyme released from atheromatous plaques into arterial blood is likely to be in an inactive form. It would appear that the inactive form of lysozyme is cleared rapidly as the raised levels in arterial blood samples are less apparent in venous samples.

Factors that may confound the interpretation of elevated lysozyme levels need to be considered. The activity of venous serum lysozyme is elevated in some cases of granulamatous diseases such as sarcoidosis (40, 41), active inflammatory bowel disease (42-45), active tuberculosis (46, 47), leprosy (46, 48), as well as myeloid leukaemia (49-51). However, these diseases can also be detected based on differential clinical diagnosis. Lysozyme is mostly (75%) catabolised by the kidneys (31). Levels of lysozyme activity did not correlate with its renal clearance in myeloid patients (50). Similarly, there was no correlation between the lysozyme levels or its activity and creatinine levels among our CAD patients. However, we cannot exclude the possibility that severe renal insufficiency may be a confounding factor in CVD patients.

In summary, circulating lysozyme levels are elevated in patients with CAD and carotid atheroma. We show that measurement of plasma levels rather than activity may be useful in the detection of active CVD. Our conclusions are based upon biological plausibility (the presence of lysozyme in the plaque, higher levels in arterial versus venous plasma, the predominance of inactive enzyme in both plaque and plasma) and the correlation between circulating levels and the severity of CAD.

### REFERENCES

1. Mackay J, Mensah G. The Atlas of Heart Disease and Stroke. World Health Organization, Centers for Disease Control and Prevention. 2004; Geneva.
2. Rosamond W, Flegal K, Furie K, et al. Heart disease and stroke statistics—2008 update: a report from the American Heart Association Statistics Committee and Stroke Statistics Subcommittee. Circulation. Jan. 29, 2008; 117(4):e25-146.
3. Braunwald's Heart Disease. 2005; Chapter 18(7th Edition):423.
4. ACC/AHA 2002 Guideline Update for the Management of Patients With Chronic Stable Angina.
5. ESC guidelines for the Management of Stable Angina Pectoris. 1999.
6. Detrano R, Gianrossi R, Froelicher V. The diagnostic accuracy of the exercise electrocardiogram: a meta-analysis of 22 years of research. Prog Cardiovasc Dis. November-December 1989; 32(3):173-206.
7. Morise A P, Diamond G A. Comparison of the sensitivity and specificity of exercise electrocardiography in biased and unbiased populations of men and women. Am Heart J. October 1995; 130(4):741-747.
8. Weiner D A, Ryan T J, McCabe C H, et al. Exercise stress testing. Correlations among history of angina, ST-segment response and prevalence of coronary-artery disease in the Coronary Artery Surgery Study (CASS). N Engl J. Med. Aug. 2, 1979; 301(5):230-235.
9. Fleischmann K E, Hunink M G, Kuntz K M, et al. Exercise echocardiography or exercise SPECT imaging? A meta-analysis of diagnostic test performance. Jama. Sep. 9, 1998; 280(10):913-920.
10. Leber A W, Knez A, von Ziegler F, et al. Quantification of obstructive and nonobstructive coronary lesions by 64-slice computed tomography: a comparative study with quantitative coronary angiography and intravascular ultrasound. J Am Coll Cardiol. Jul. 5, 2005; 46(1):147-154.
11. Raff G L, Gallagher M J, O'Neill W W, et al. Diagnostic accuracy of noninvasive coronary angiography using 64-slice spiral computed tomography. J Am Coll Cardiol. Aug. 2, 2005; 46(3):552-557.
12. Gerber T C, Kuzo R S, Lane G E, et al. Image quality in a standardized algorithm for minimally invasive coronary angiography with multislice spiral computed tomography. J Comput Assist Tomogr. January-February 2003; 27(1):62-69.
13. Samani N J, Thompson J R, O'Toole L, et al. A meta-analysis of the association of the deletion allele of the angiotensin-converting enzyme gene with myocardial infarction. Circulation. Aug. 15, 1996; 94(4):708-712.
14. Oram J F, Heinecke J W. ATP-binding cassette transporter A1: a cell cholesterol exporter that protects against cardiovascular disease. Physiol Rev. October 2005; 85(4):1343-1372.
15. Stenina O I, Byzova T V, Adams J C, et al. Coronary artery disease and the thrombospondin single nucleotide polymorphisms. Int J Biochem Cell Biol. June 2004; 36(6):1013-1030.
16. Pecoits-Filho R, Stenvinkel P, Marchlewska A, et al. A functional variant of the myeloperoxidase gene is associated with cardiovascular disease in end-stage renal disease patients. Kidney Int Suppl. May 2003(84):S172-176.
17. Vadseth C, Souza J M, Thomson L, et al. Pro-thrombotic state induced by post-translational modification of fibrinogen by reactive nitrogen species. J Biol. Chem. Mar. 5, 2004; 279(10):8820-8826.
18. Burgess-Cassler A, Johansen J J, Kendrick N C. Two-dimensional gel analysis of serum apolipoprotein A-I isoforms: preliminary analysis suggests altered ratios in individuals with heart disease. Appl Theor Electrophor. 1992; 3(1):41-45.
19. Takemoto M, Liao J K. Pleiotropic effects of 3-hydroxy-3-methylglutaryl coenzyme a reductase inhibitors. Arterioscler Thromb Vasc Biol. November 2001; 21(11):1712-1719.
20. Christenson R H, Azzazy H M. Biochemical markers of the acute coronary syndromes. Clin Chem. August 1998; 44(8 Pt 2):1855-1864.
21. Zouridakis E, Avanzas P, Arroyo-Espliguero R, et al. Markers of inflammation and rapid coronary artery disease progression in patients with stable angina pectoris. Circulation. Sep. 28, 2004; 110(13):1747-1753.
22. Ndrepepa G, Braun S, Niemoller K, et al. Prognostic value of N-terminal pro-brain natriuretic peptide in patients with chronic stable angina. Circulation. Oct. 4, 2005; 112(14):2102-2107.
23. Ridker P M, Buring J E, Shih J, et al. Prospective study of C-reactive protein and the risk of future cardiovascular events among apparently healthy women. Circulation. Aug. 25, 1998; 98(8):731-733.
24. Abdul-Salam V B, Paul G A, Ali J O, et al. Identification of plasma protein biomarkers associated with idiopathic pulmonary arterial hypertension. Proteomics. April 2006; 6(7):2286-2294.
25. Blanco-Colio L M, Martin-Ventura J L, Vivanco F, et al. Biology of atherosclerotic plaques: what we are learning from proteomic analysis. Cardiovasc Res. Oct. 1, 2006; 72(1):18-29.
26. Zimmerli L U, Schiffer E, Zurbig P, et al. Urinary proteomic biomarkers in coronary artery disease. Mol Cell Proteomics. February 2008; 7(2):290-298.
27. Donahue M P, Rose K, Hochstrasser D, et al. Discovery of proteins related to coronary artery disease using industrial-scale proteomics analysis of pooled plasma. Am Heart J. September 2006; 152(3):478-485.
28. Inoue T, Eguchi Y, Matsumoto T, et al. Lipocalin-type prostaglandin D synthase is to a powerful biomarker for severity of stable coronary artery disease. Atherosclerosis. Mar. 16, 2008.
29. Pu L J, Lu L, Shen W F, et al. Increased serum glycated albumin level is associated with the presence and severity of coronary artery disease in type 2 diabetic patients. Circ J. July 2007; 71 (7):1067-1073.
30. Walter M F, Jacob R F, Bjork R E, et al. Circulating lipid hydroperoxides predict cardiovascular events in patients with stable coronary artery disease: the PREVENT study. J Am Coll Cardiol. Mar. 25, 2008; 51(12):1196-1202.
31. Hansen N E, Karle H, Andersen V, et al. Lysozyme turnover in man. J Clin Invest. May 1972; 51(5):1146-1155.
32. Schaffner T, Taylor K, Bartucci E J, et al. Arterial foam cells with distinctive immunomorphologic and histochemical features of macrophages. Am J. Pathol. July 1980; 100(1):57-80.
33. Bagnato C, Thumar J, Mayya V, et al. Proteomics analysis of human coronary atherosclerotic plaque: a feasibility study of direct tissue proteomics by liquid chromatography and tandem mass spectrometry. Mol Cell Proteomics. June 2007; 6(6)1088-1102.
34. Naito M, Nomura H, Esaki T, et al. Characteristics of macrophage-derived foam cells isolated from atherosclerotic lesions of rabbits. Atherosclerosis. December 1997; 135(2):241-247.
35. Vissers M C, Winterbourn C C. Myeloperoxidase-dependent oxidative inactivation of neutrophil neutral proteinases and microbicidal enzymes. Biochem J. Jul. 1, 1987; 245(1):277-280.
36. Naskalski J W. Oxidative modification of protein structures under the action of myeloperoxidase and the hydrogen peroxide and chloride system. Ann Biol Clin (Paris). 1994; 52(6):451-456.
37. Heinecke J W. Mechanisms of oxidative damage by myeloperoxidase in atherosclerosis and other inflammatory disorders. J Lab Clin Med. April 1999; 133(4):321-325.
38. Karlsson H, Leanderson P, Tagesson C, et al. Lipoproteomics I: mapping of proteins in low-density lipoprotein using two-dimensional gel electrophoresis and mass spectrometry. Proteomics. February 2005; 5(2):551-565.
39. Brody J I, Pickering N J, Fink G B. Immunocytochemical features of obstructed saphenous vein coronary artery bypass grafts. J Clin Pathol. May 1989; 42(5):477-482.
40. Tomita H, Sato S, Matsuda R, et al. Serum lysozyme levels and clinical features of sarcoidosis. Lung. 1999; 177(3):161-167.
41. Turton C W, Grundy E, Firth G, et al. Value of measuring serum angiotensin I converting enzyme and serum lysozyme in the management of sarcoidosis. Thorax. February 1979; 34(1):57-62.
42. Klass H J, Neale G. Serum and faecal lysozyme in inflammatory bowel disease. Gut. March 1978; 19(3):233-239.
43. Mallas E, Terry J M, Asquith P, et al. Serum lysozyme in inflammatory bowel and coeliac disease. J Clin Pathol. July 1976; 29(7):598-600.
44. Dronfield M W, Langman M J. Serum lysozyme in inflammatory bowel disease. Gut. December 1975; 16(12):985-987.
45. Peeters T L, Vantrappen G, Geboes K. Serum lysozyme levels in Crohn's disease and ulcerative colitis. Gut. April 1976; 17(4):300-305.
46. Near K A, Lefford M J. Use of serum antibody and lysozyme levels for diagnosis of leprosy and tuberculosis. J Clin Microbiol. May 1992; 30(5):1105-1110.
47. Selvaraj P, Kannapiran M, Reetha A M, et al. HLA-DR2 phenotype and plasma lysozyme, beta-glucuronidase and acid phosphatase levels in pulmonary tuberculosis. Int J Tuberc Lung Dis. June 1997; 1(3):265-269.
48. Rea T H, Taylor C R. Serum and tissue lysozyme in leprosy. Infect Immun. December 1977; 18(3):847-856.
49. Sexton C, Buss D, Powell B, et al. Usefulness and limitations of serum and urine lysozyme levels in the classification of acute myeloid leukemia: an analysis of 208 cases. Leuk Res. June 1996; 20(6):467-472.
50. Pruzanski W, Platts M E. Serum and urinary proteins, lysozyme (muramidase), and renal dysfunction in mono- and myelomonocytic leukemia. J Clin Invest. September 1970; 49(9):1694-1708.
51. Astrom M, Bodin L, Hornsten P, et al. Evidence for a bimodal relation between serum lysozyme and prognosis in 232 patients with acute myeloid leukaemia. Eur J. Haematol. January 2003; 70(1):26-33.
52. Henke C E, Henke G, Elveback L R, et al. The epidemiology of sarcoidosis in Rochester, Minn.: a population-based study of incidence and survival. Am J Epidemiol. May 1986; 123(5):840-845.
53. Lawlor D A, Smith G D, Leon D A, et al. Secular trends in mortality by stroke subtype in the 20th century: a retrospective analysis. Lancet. Dec. 7, 2002; 360(9348): 1818-1823.
54. Rothwell P M, Coull A J, Silver L E, et al. Population-based study of event-rate, incidence, case fatality, and mortality for all acute vascular events in all arterial territories (Oxford Vascular Study). Lancet. Nov. 19, 2005; 366(9499):1773-1783.
55. Rubin G P, Hungin A P, Kelly P J, et al. Inflammatory bowel disease: epidemiology and management in an English general practice population. Aliment Pharmacol Ther. December 2000; 14(12):1553-1559.
56. Anderson J L. Lipoprotein-associated phospholipase A2: an independent predictor of coronary artery disease events in primary and secondary prevention. Am J. Cardiol. Jun. 16, 2008; 101(12A):23F-33F.
57. Ridker P M. C-reactive protein and the prediction of cardiovascular events among those at intermediate risk: moving an inflammatory hypothesis toward consensus. Am Coll Cardiol. May 29, 2007; 49(21):2129-2138.
58. de Lemos J A, Zirlik A, Schonbeck U, et al. Associations between soluble CD40 ligand, atherosclerosis risk factors, and subclinical atherosclerosis: results from the Dallas Heart Study. Arterioscler Thromb Vasc Biol. October 2005; 25(10):2192-2196.
59. Cavusoglu E, Komecki E, Sobocka M B, et al. Association of plasma levels of F11 receptor/junctional adhesion molecule-A (F11R/JAM-A) with human atherosclerosis. J Am Coll Cardiol. Oct. 30, 2007; 50(18):1768-1776.
60. Asanuma Y, Chung C P, Oeser A, et al. Serum osteoprotegerin is increased and independently associated with coronary-artery atherosclerosis in patients with rheumatoid arthritis. Atherosclerosis. December 2007; 195(2):e135-141.
61. Koenig W, Khuseyinova N. Biomarkers of atherosclerotic plaque instability and rupture. Arterioscler Thromb Vasc Biol. January 2007; 27(1):15-26.
62. Gibbons R J, Abrams J, Chatterjee K, Daley J, Deedwania P C, Douglas J S, Ferguson T B, Jr., Fihn S D, Fraker T D, Jr., Gardin J M, O'Rourke R A, Pasternak R C, Williams S V, Gibbons R J, Alpert J S, Antman E M, Hiratzka L F, Fuster V, Faxon D P, Gregoratos G, Jacobs A K, Smith S C, Jr. ACC/AHA 2002 guideline update for the management of patients with chronic stable angina—summary article: a report of the American College of Cardiology/American Heart Association Task Force on Practice Guidelines (Committee on the Management of Patients With Chronic Stable Angina). Circulation. 2003; 107:149-158.

### APPENDIX 1

### Details of Biomedical Technologies Inc. HUMAN LYSOZYME EIA KIT

The kit is for the measurement of human lysozyme in serum, plasma, urine, tears, saliva, and other body fluids.

### Introduction

Lysozyme (muramidase) hydrolyses principally the B-1,4 glucosidic linkages between n-acetylmuramic acid and n-acetylglucosamine occurring in the mucopeptide cell wall of some microorganisms. The enzyme has widespread distribution in animals and plants. In normal humans, relatively large concentrations of lysozymes are present in serum/plasma, amniotic fluid, saliva and tears with lessor quantities in urine, bile and cerebrospinal fluid. Elevated concentrations of urine and serum lysozyme have been reported in several human diseases and conditions including some leukemias, tuberculosis, megaloblastic anemias, acute bacterial infections, ulcerative colitis, severe renal insufficiency, pyelonephritis and nephrosis.

### Principal of the Assay

This is a sandwich ELISA assay for human lysozyme. A monoclonal antibody specific for lysozyme is bound to polystyrene wells. After an incubation with sample, the plate is washed followed by an incubation with a second human lysozyme specific antibody (sheep polyclonal). Detection is achieved by a third incubation using a Horseradish Peroxidase conjugate of Donkey anti-Goat (sheep) IgG and subsequent enzyme assay. Concentration of human lysozyme is proportional to color development. Exact levels are obtained from a standard curve using purified human lysozyme.

### REFERENCES

A. Hankiewiz, J. and Swierczuk, E. (1974) Lysozymes in Human Body Fluids. Clinica Chemica Acta, 57: 205-209.
B. Meyor, K., Gelhorn, A., Prudden, J. F., et al (1948) Lysozyme Activity in Ulcerative Alimentary Disease. American Journal of Medicine, 5: 496-502.
C. Prockup, D. J. and Davidson, W. D. (1964) A Study of Urinary and Serum Lysozyme in Patients with Renal Disease, New England Journal of Medicine, 270: 269.
D. Davis, C. S. (Apr. 5, 1971) Diagnostic Value of Muramidase. Laboratory Medicine, 51-54.

### Reagents: Description and Preparation

Store all reagents at 4° C. up to 6 months except as noted (*see storage exception)

### CAUTION: DO NOT USE AZIDE, OR AZIDE CONTAINING SAMPLES.

1. Phosphate-Saline Concentrate BT-492. One 100 ml bottle. Transfer contents to a graduated cylinder, and bring volume up to 500 ml with deionized water. Use this buffer for the preparation of standards, samples and for washing the plate.
2. Human Lysozyme Standard, BT-631. One vial, 1000 ng lyophilized. Reconstitute with exactly 1 ml deionized water. Use this stock solution for making working standards. Store the stock solution at −20° C. up to 2 weeks.
3. Lysozyme Antiserum, BT-632. One 12 ml vial.
4. Conjugate Buffer, BT-633. One 12 ml vial.
5. Donkey anti-Goat IgG Peroxidase Conjugate, BT-495. Glycerol solution. One vial.
*Store at −20° C. Dilute 1/800 (15 ul for 12 ml) using Conjugate Buffer. NOTE: only prepare enough solution for one day's use. Discard excess solution.
6. Peroxidase Substrate TMB, BT-497. One 6 ml vial.
7. Hydrogen Peroxide Solution, BT-498. One 6 ml vial.
8. Stop Solution, BT-499. One 12 ml vial.
9. One 96 well plate (8 well removable strips) coated with a monoclonal human lysozyme antibody.
10. Human Lysozyme Control (Urine, Lyophilized), BT-634. Reconstitute with 0.5 ml-1.0 ml phosphate-saline buffer. Cap, mix end-over-end until the solids are dissolved. Store the solution at −20° C. for one month.

### Other Supplies Required

1. Elisa Plate Reader which can measure absorbance at 450 nm.
2. Pipettes: micropipettes 5-1000 ul.
3. A plate washer is recommended for washing.

### 4. A 37° C. Incubator.

5. Deionized water.

### Precautions

Some components of this kit contain isothiazolones (5 ppm) as a preservative. Stop solution contains hydrochloric and phosphoric acids. Keep all materials away from the skin and eyes.

### Sample Preparation

Collect samples in leak proof containers. Store serum (plasma), urine and body fluids (eg. saliva and tears) at 4° C. for 2 days or 2 weeks at −20° C. Thaw and keep on ice until ready for use. Caution: Samples must not contain azides. Most samples require dilution with Phosphate-Saline Buffer: Urine, 1/10-1/50; serum (plasma), saliva, at least 1/2000; tears, approximately 1/10,000.

### Range of Normal Values Reported

Serum (Plasma) 4-13 ug/ml
Urine 0-2 ug/ml
Saliva 4-13 ug/ml
Tears >300 ug/ml

### Values Observed at BTI

Urine 20-300 ng/ml
Saliva 100 ug-200 ug/ml
Serum 3-10 ug/ml

### Standards

Prepare a set of standards from the 1000 ng/ml stock in the range of 0.5 to 50 ng/ml using diluted Phosphate-Saline Buffer. For example:

| | | | | Concentration |
|---|---|---|---|---|
| Standard # | ml of Std. | | ml of Buffer | ng/ml |
| 1. | .05 | stock | 0.95 | 50 |
| 2. | 0.5 | std. 1 | 0.5 | 25 |
| 3. | 0.5 | std. 2 | 0.5 | 12.5 |
| 4. | 0.5 | std. 3 | 0.5 | 6.25 |
| 5. | 0.5 | std. 4 | 0.5 | 3.125 |
| 6. | 0.5 | std. 5 | 0.5 | 1.56 |
| 7. | 0.5 | std. 6 | 0.5 | 0.78 |

Store the stock solution frozen (−20° C.). Discard all working standards.

### Assay Procedure

### CAUTION: KEEP AZIDES AWAY FROM ALL SOLUTIONS AND SAMPLES

All Reagents must be at room temperature prior to use.
1. Prepare reagents, standards and samples as described on pages 2 and 3 respectively.
2. Remove microtiter plate from resealable bag. Strips not used should be removed from the frame, resealed in the bag and stored at 4° C. for future use.
3. Pipet 100 ul of wash buffer (Blank), standards, samples and controls into designated duplicate wells. Cover tightly with plastic seal and incubate at room temperature for 2 hours.
4. Aspirate wells completely and wash the plate 3 times with Phosphate-Saline wash buffer. Complete removal of wash buffer after each wash is important for good reproducibility. Add 100 ul of the Lysozyme Antiserum to each well. Cover tightly, incubate at room temperature for 1 hour.
5. Wash as in step 4. Add 100 ul of the diluted Donkey anti-Goat IgG Peroxidase to each well. Incubate at room temperature for 1 hour.
6. Mix one volume of TMB solution (BT-497) with one volume of Hydrogen Peroxide solution (BT-498) and put aside. Only mix an amount sufficient for the number of wells in use. Wash the plate as in step 4. Immediately add 100 ul of substrate mix to all wells and incubate at room temperature, in the dark for 15 minutes.
7. Add 100 ul of Stop Solution to all wells, swirl and measure absorbance at 450 nm within 15 minutes.

### Calculation of Results

Average duplicates for all determinations. Subtract the Blank from all average readings. Plot net optical density of the standards vs. log of the concentration of each. Draw the best curve. Obtain concentration of each unknown from this standard curve. Always generate a standard curve for each new assay.

### Specifications

Sample size: 100 ul
Assay time: 4.25 hrs.
Sensitivity: 0.78 ng/ml
Working range: 0.78-50 ng/ml
Intraassay variation: 5.3%
Interassay variation: 7%
Recovery (urine): 105%
Typical Data (Do not use for determination of Unknowns)

| ID | A 450 nm | Average-Blank |
|---|---|---|
| Blank, 0 ng/ml | .213 | |
| Blank, 0 ng/ml | .202 | |
| 0.78 ng/ml | .393 | |
| 0.78 ng/ml | .424 | .201 |
| 1.5 ng/ml | .656 | |
| 1.5 ng/ml | .566 | .404 |
| 3.12 ng/ml | .889 | |
| 3.12 ng/ml | .874 | .674 |
| 6.25 ng/ml | 1.234 | |
| 6.25 ng/ml | 1.238 | 1.029 |
| 12.5 ng/ml | 1.778 | |
| 12.5 ng/ml | 1.739 | 1.555 |
| 25 ng/ml | 2.176 | |
| 25 ng/ml | 2.285 | 2.023 |
| 50 ng/ml | 2.400 | |
| 50 ng/ml | 2.546 | 2.266 |

A typical standard curve is shown in FIG. 11.

1. A method of diagnosing atheroma in an individual, the method comprising:
providing a sample from the individual;
determining the level of lysozyme in the sample; and
assessing whether the level is indicative of atheroma in the individual.

2. A method of prognosing atheroma in an individual who has atheroma, the method comprising:
providing a sample from the individual;
determining the level of lysozyme in the sample; and
assessing whether the level is indicative of a particular outcome for the individual.

3. The method according to claim 1, wherein the atheroma is in any one or more of the aorta, a coronary artery, a renal artery, a neck artery, a cerebral artery, a limb artery or a visceral artery.

4. The method according to claim 1 wherein a level of lysozyme in the sample of greater than 1 standard deviation (SD) above the mean level of lysozyme in a healthy population is indicative of atheroma.

5. (canceled)

6. The method according to claim 3, wherein a level of lysozyme in the sample greater than 1.5 μg/mL is indicative of atheroma.

7. The method according to claim 1 wherein the sample is a blood, serum or plasma sample.

8. The method according to claim 1, wherein the sample is arterial blood, serum or plasma.

9. The method according to claim 8, wherein a level of lysozyme in the sample greater than 1.5 μg/mL is indicative of atheroma in a coronary artery wherein at least one vessel is occluded by more than two-thirds when assessed by angiography.

10-13. (canceled)

14. The method according to claim 2, wherein a level of lysozyme in the sample which of greater than 2 standard deviations (SD) above the mean level of lysozyme in a population of atheroma individuals who fare well is indicative of a poor outcome.

15. The method according to claim 2, wherein a level of lysozyme in the sample greater than 1.5 μg/mL is indicative of a poor outcome.

16. The method according to claim 14, wherein the sample is a blood, serum or plasma sample.

17-19. (canceled)

20. The method according to any of claim 1, further comprising:
determining the level of one or more further atheroma markers in the sample from the individual; and
assessing whether the level of said further marker or markers is indicative of atheroma in the individual,
wherein the further marker is any of cholesterol, lipocalin-type prostaglandin D, glycated albumin myeloperoxidase, and lipid peroxidase.

21. The method of claim 2, further comprising:
determining the level of one or more further atheroma markers in the sample from the individual; and
assessing whether the levels of said further marker or markers is indicative of a particular outcome for the individual,
wherein the further marker is any of cholesterol, lipocalin-type prostaglandin D, glycated albumin, myeloperoxidase, and lipid peroxidase.

22-37. (canceled)

38. A kit of parts comprising a reagent that selectively identifies lysozyme and a reagent which selectively identifies a further marker of atheroma, wherein the further marker of atheroma is any of cholesterol, lipocalin-type prostaglandin D, glycated albumin, myeloperoxidase and lipid peroxidase.

39-44. (canceled)

45. A method of assessing whether a compound has an effect on atheroma in a individual, the method comprising:
administering to the individual the compound, and
determining the effect of the compound on the level of lysozyme in the individual.

46. The method according to claim 45 wherein the level of lysozyme in the individual's blood, plasma or serum is determined.

47. The method according to claim 45 further comprising:
determining the level of a further atheroma marker in the individual.

48. The method according to claim 47 wherein the further atheroma marker is any of cholesterol, lipocalin-type prostaglandin D, glycated albumin, myeloperoxidase or lipid peroxidise.

49-50. (canceled)

51. A method of combating atheroma in an individual, the method comprising:
diagnosing atheroma in the individual according to the method of claim 1; and
treating the atheroma,
wherein the atheroma is in any one or more of the aorta, a coronary artery, a renal artery, a neck artery, a cerebral artery, a limb artery, and a visceral artery.

52. The method according to claim 51, wherein treating the atheroma comprises administering at least one of a statin; a lipid lowering drug; a drug which modifies the angiotensin system, an angiotensin receptor blocker or a rennin antagonist; an anti-platelet; an anticoagulant drug; and a disease modifying agent for diabetes to the individual.

53-57. (canceled)

58. The method of claim 2, wherein the atheroma is in any one or more of the aorta, a coronary artery, a renal artery, a neck artery, a cerebral artery, a limb artery, and a visceral artery.
